(19) United States
(12) Patent Application Publication
(10) Pub. No.: US 2017/0007853 A1
(43) Pub. Date: Jan. 12, 2017

(54) PHYSIOLOGICAL MONITORING FOR ULTRASOUND THERAPY

(71) Applicant: Medtronic, Inc., Minneapolis, MN (US)

(72) Inventors: Jamu K. Alford, Ham Lake, MN (US); Erik R. Scott, Maple Grove, MN (US); John D. Welter, Santa Rosa, CA (US); John R. LaLonde, Lake Elmo, MN (US); Yohan Kim, Fridley, MN (US)

(21) Appl. No.: 15/204,430

(22) Filed: Jul. 7, 2016

Related U.S. Application Data
(60) Provisional application No. 62/191,135, filed on Jul. 10, 2015.
(60) Provisional application No. 62/236,058, filed on Oct. 1, 2015.

(51) Int. Cl. A61N 7/00; A61B 8/04; A61B 5/00; A61B 5/01; A61B 5/0205; A61B 5/145; A61B 8/02; A61B 5/11

(52) U.S. Cl. CPC A61N 7/00; A61B 8/02; A61B 8/04; A61B 5/1118; A61B 5/01; A61B 5/02055; A61B 5/14542; A61B 5/14546; A61B 5/4848; A61N 2007/0021; A61B 5/0816

(57) ABSTRACT

In some examples, a system includes a flexible ultrasound device configured to be attached to an external surface of a patient proximate to an organ of the patient to deliver ultrasound configured to modulate nerve tissue of the patient at the organ. The system further comprises one or more sensors configured to sense one or more physiological parameters indicative of at least one of a symptom treatable by, or a side effect of, the neuromodulation, and processing circuitry configured to control the delivery of ultrasound during an ambulatory period of the patient, and monitor the least one of the symptom or the side effect during the ambulatory period, based on the one or more physiological parameters. The organ may be the spleen and the ultrasound may at least one of regulate the autoimmune system of the patient, or reduce an inflammation response of the patient.

This application claims the benefit of U.S. Provisional Application Ser. No. 62/191,135, filed Jul. 10, 2015, and U.S. Provisional Application Ser. No. 62/236,058, filed Oct. 1, 2015, the entire contents of which are incorporated herein by reference.

### TECHNICAL FIELD

This disclosure relates to physiological monitoring and delivery of ultrasound for diagnosis and/or therapy.

### BACKGROUND

Delivery of ultrasound involves delivering sound waves with frequencies higher than the upper audible limit of human hearing. Delivery of ultrasound is performed for diagnostic imaging, e.g., to visualize internal body structures such as tendons, muscles, joints, vessels, and internal organs. Ultrasound images are made delivering ultrasound, e.g., pulses of ultrasound, into tissue using one or more ultrasound transducers. The sound echoes, or reflects, off the tissue, with different tissues having different characteristics reflecting and diffracting the sound differently. The reflected and diffracted sound is sensed by one or more ultrasound transducers.

Ultrasound has also been delivered to patients for therapeutic purposes. For example, ultrasound has been delivered to promote healing and/or blood flow. As another example, ultrasound has been delivered to modify or destroy problematic tissue, such as tumors. It has also been proposed to modulate the nervous system using ultrasound. In each of these cases, the therapeutic effect of ultrasound may be due to mechanical forces, acoustic streaming, heating and/or cavitation of the tissue.

Delivery of ultrasound for medical purposes often involves a relatively-large, cart-based piece of equipment that includes, for example, circuitry for generating and sensing ultrasound signals, processing circuitry, a user interface, and an internal power source and/or the ability to be plugged to AC mains power. A probe that includes the one or more ultrasound transducers may be connected to ultrasound device by a cable.

### SUMMARY

This disclosure is related to devices, systems, and techniques for delivery of ultrasound for diagnosis and/or therapy. For example, a system and/or device may be configured to monitor physiological parameters indicative of one or more symptoms or physiological activities, a patient's response to ultrasound therapy delivery, or the efficacy of another therapy. The system may perform monitoring on an acute basis or for long-term monitoring, and the system may monitor physiological parameters via ultrasound imaging from a wearable ultrasound device, or one or more sensors configured to detect one or more vital signs of the patient, and/or biological components such as cytokines and/or blood constituents. Although the system may be used only for monitoring in some examples, in some examples the system may use detected physiological parameters as feedback mechanisms to control ultrasound signals delivered to the patient from the wearable ultrasound device. In this manner, systems described herein may deliver ultrasound therapy to control inflammation and immune response, modulate physiological activity, or even assist other therapies delivered to the patient such as drug therapies.

In one example, a system comprises a flexible ultrasound device configured to be attached to an external surface of a patient proximate to an organ of the patient. The flexible ultrasound device comprises a flexible interconnect element, a plurality of ultrasound transducers connected to the flexible interconnect element, one or more power sources connected to the flexible interconnect element, and signal generation circuitry powered by the one or more power sources and connected to the flexible interconnect element, wherein the signal generation circuitry is configured to generate a signal that drives one or more of the ultrasound transducers to deliver an ultrasound signal to the organ, the ultrasound signal configured to modulate nerve tissue of the patient at the organ. The system further comprises one or more sensors configured to sense one or more physiological parameters of the patient, the one or more physiological parameters indicative of at least one of a symptom treatable by the modulation of the nerve tissue of the patient at the organ, or a side effect of the modulation of the nerve tissue of the patient at the organ. The system further comprises processing circuitry configured to control the signal generation circuitry to generate the signal and drive the one or more ultrasound transducers during an ambulatory period of the patient to modulate the nerve tissue at the organ, and monitor the least one of the symptom or the side effect during the ambulatory period based on the one or more physiological parameters.

In another example a method of delivering ultrasound with a flexible ultrasound device configured to be attached to an external surface of a patient proximate to an organ of the patient comprising delivering ultrasound from the flexible ultrasound device to the organ during an ambulatory period of the patient, the ultrasound configured to modulate nerve tissue of the patient at the organ, sensing, via one or more sensors, one or more physiological parameters of the patient during the ambulatory period, the one or more physiological parameters indicative of at least one of a symptom treatable by the modulation of the nerve tissue of the patient at the organ, or a side effect of the modulation of the nerve tissue of the patient at the organ, and monitoring, via processing circuitry, the least one of the symptom or the side effect during the ambulatory period based on the one or more physiological parameters.

In another example, a system for delivering ultrasound with a flexible ultrasound device configured to be attached to an external surface of a patient proximate to an organ of the patient comprises means for delivering ultrasound from the flexible ultrasound device to the organ during an ambulatory period of the patient, the ultrasound configured to modulate nerve tissue of the patient at the organ, means for sensing one or more physiological parameters of the patient during the ambulatory period, the one or more physiological parameters indicative of at least one of a symptom treatable by the modulation of the nerve tissue of the patient at the organ, or a side effect of the modulation of the nerve tissue of the patient at the organ, and means for monitoring the least one of the symptom or the side effect during the ambulatory period based on the one or more physiological parameters.

In another example, a computer-readable storage medium comprising program instructions that, when executed by processing circuitry, cause the processing circuitry to control a flexible ultrasound device to deliver ultrasound to an organ of a patient during an ambulatory period of the patient, the ultrasound configured to modulate nerve tissue of the patient at the organ, and the flexible ultrasound device configured to be attached to an external surface of the patient proximate to the organ of the patient, control one or more sensors to sensing one or more physiological parameters of the patient during the ambulatory period, the one or more physiological parameters indicative of at least one of a symptom treatable by the modulation of the nerve tissue of the patient at the organ, or a side effect of the modulation of the nerve tissue of the patient at the organ, and monitor the least one of the symptom or the side effect during the ambulatory period based on the one or more physiological parameters.

In another example, this disclosure is directed to a method that includes generating, by sensing circuitry of a wearable ultrasound device, ultrasound imaging signals indicative of a physiological parameter over a period of time, the wearable ultrasound device comprising a flexible interconnect element, a plurality of ultrasound transducers connected to the flexible interconnect element, one or more power sources connected to the flexible interconnect element, signal generation circuitry powered by the one or more power sources and connected to the flexible interconnect element, wherein the signal generation circuitry is configured to generate a drive signal that drives one or more ultrasound transducers of the ultrasound transducers to deliver an ultrasound signal to target anatomy; and the sensing circuitry, wherein the sensing circuitry is connected to one or more of the plurality of ultrasound transducers and the flexible interconnect element, and wherein, for at least one ultrasound transducer of the plurality of ultrasound transducers, the sensing circuitry is configured to generate the imaging signals as a function of reflected ultrasound sensed by the at least one ultrasound transducer, determining, based on the ultrasound imaging signals, that a value of the physiological parameter has exceeded a threshold, and outputting an indication of the determination.

In another example, this disclosure is directed to a wearable ultrasound device that includes a flexible interconnect element, a plurality of ultrasound transducers connected to the flexible interconnect element, one or more power sources connected to the flexible interconnect element, signal generation circuitry powered by the one or more power sources and connected to the flexible interconnect element, wherein the signal generation circuitry is configured to generate a drive signal that drives one or more ultrasound transducers of the ultrasound transducers to deliver an ultrasound signal to target anatomy, sensing circuitry connected to one or more of the plurality of ultrasound transducers and the flexible interconnect element and configured to generate ultrasound imaging signals indicative of a physiological parameter over a period of time, and processing circuitry configured to control the signal generation circuitry and the sensing circuitry, determine, based on the ultrasound imaging signals, that a value of the physiological parameter has exceeded a threshold, and output an indication of the determination.

In another example, this disclosure is directed to a system comprising means for performing any of the methods described in this disclosure.

In another example, this disclosure is directed to a computer-readable storage medium comprising instructions that, when executed by processing circuitry, cause the processing circuitry to perform any of the methods described in this disclosure.

The details of one or more examples of this disclosure may be set forth in the accompanying drawings and the description below. Other features, objects, and advantages of this disclosure may be apparent from the description and drawings, and from the claims.

### BRIEF DESCRIPTION OF DRAWINGS

FIG. 1 is a conceptual diagram illustrating an example system for delivering ultrasound to a patient and monitoring the patient.

FIGS. 2A and 2B are top-view and side-view diagrams, respectively, illustrating an example wearable ultrasound device.

FIG. 3 is a top-view diagram illustrating another example wearable ultrasound device.

FIG. 4 is a top-view diagram illustrating another example wearable ultrasound device that includes a plurality of physiological parameter sensors.

FIG. 5 is a functional block diagram illustrating an example configuration of a wearable ultrasound device.

FIG. 6 is a functional block diagram illustrating an example configuration of an interface device configured to communicate with a wearable ultrasound device.

FIG. 7 is a block diagram illustrating another example system for delivering ultrasound to a patient and monitoring the patient.

FIG. 8 is a flow diagram illustrating an example method for delivering ultrasound to an organ and monitoring a patient during the delivery of the ultrasound.

FIG. 9 is a flow diagram illustrating an example method for delivering ultrasound to the spleen and monitoring a patient during the delivery of the ultrasound.

FIG. 10 is a flow diagram illustrating an example method for determining changes to a physiological parameter based on ultrasound imaging signals.

### DETAILED DESCRIPTION

This disclosure is related to devices, systems, and techniques for delivery of ultrasound for diagnosis and/or therapy. Ultrasound imaging of patient anatomy may be used to non-invasively identify tissue structures and physiological states (e.g., cardiac tissue and blood flow, tissue elasticity), and some ultrasound signals may be used to modify patient anatomy is some situations (e.g., break up kidney stones or ablate tumors). However, these procedures and typically performed at a health care clinic and involve a large cart-based ultrasound generator and hand-held transducer that is operated by a trained clinician or physician. Moreover, these large ultrasound devices make long-term monitoring or chronic therapy delivery unfeasible.

As described herein, a wearable ultrasound device may be configured to perform a variety of different functions related to a patient. For example, a system may include the wearable ultrasound device and be configured to monitor physiological parameters indicative of one or more symptoms or physiological activities, a patient's response to ultrasound therapy delivery, or the efficacy of another therapy. The system may perform monitoring on an acute basis and/or on a long-term basis, e.g., longitudinal monitoring. In addition, the system may monitor physiological parameters via ultrasound imaging from the wearable ultrasound device (e.g., using an array of ultrasound transducers), one or more sensors configured to detect one or more vital signs of the patient (e.g., sensors configured to obtain a temperature, heart rate, breathing rate, blood oxygenation, patient activity, or even blood samples), and/or biological components such as cytokines and/or blood constituents. In some examples, the system may even generate alerts when one or more monitored physiological parameters exceed a threshold or otherwise indicate that the patient may benefit from medical treatment and/or a change in the currently delivered therapy.

Although the system may be used only for monitoring in some examples, in some examples the system may use these detected physiological parameters as feedback mechanisms to control ultrasound signals delivered to the patient from the wearable ultrasound device. In one example, systems described herein may include one or more wearable ultrasound devices configured to deliver ultrasound therapy that modulates (e.g., increases or decreases) inflammation and immune response from the patient. The system may utilize ultrasound signals delivered from the wearable ultrasound device to modulate physiological activity (e.g., nerve or organ function) or even assist other therapies delivered to the patient such as drug therapies (e.g., chemotherapy delivery to treat cancer). The system may provide or support other therapies including accelerated wound healing, activation of pharmaceuticals or contrast agents, heating of tissue directed improve and/or activate chemotherapy treatments, local hyperthermia immunomodulation, clot lysis, stem cell homing, vasoconstriction, amplification of cancer biomarkers and sonodynamic therapy.

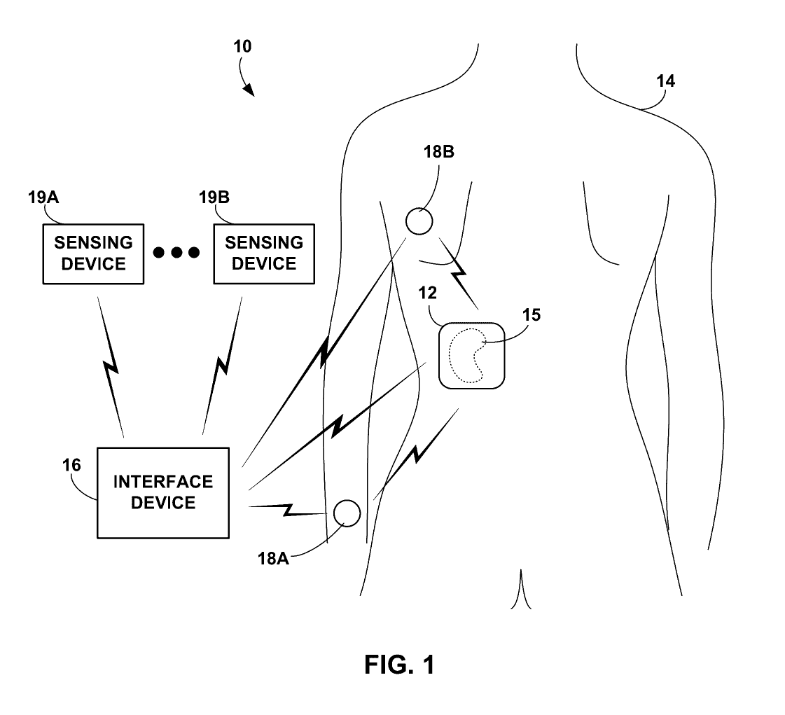

FIG. 1 is a conceptual diagram illustrating an example system 10 for delivering ultrasound to a patient 14 and monitoring patient 14. As illustrated in FIG. 1, system 10 includes a wearable ultrasound device 12 attached to patient 14. As will be described in greater detail below, wearable ultrasound device 12 may include a plurality of ultrasound transducers, signal generation circuitry configured to drive the plurality of ultrasound transducers, one or more power sources configured to power the signal generation circuitry, and one or more processors or other processing circuitry configured to control the signal generation circuitry.

In some examples, the components of wearable ultrasound device 12 may be configured, e.g., constructed and arranged, such that wearable ultrasound device 12 is flexible. In some examples, wearable ultrasound device 12 is flexible such that it conforms to a surface of patient 14 on which the wearable ultrasound device is attached. Wearable ultrasound device 12 may be used, and attached to patient 14, for time periods as brief as a few minutes to as long as several months. The flexibility of wearable ultrasound device 12 may increase the comfort of patient 14.

System 10 may be used for diagnostic and/or therapeutic applications, and may include an attachment element configured to maintain the position of the ultrasound transducers of wearable ultrasound device 12 relative to a treatment or diagnostic area of patient 14. In some examples, wearable ultrasound device 12 may include an adhesive layer as an attachment element for attaching the device to patient 14. In addition to, or instead of the adhesive layer, in some examples, an attachment element may comprise a strap or garment.

Wearable ultrasound device 12 may deliver ultrasound to patient 14 for diagnostic imaging. In some examples, wearable ultrasound device 12 may deliver ultrasound to patient 14 for therapeutic purposes, such as neuromodulation. In some examples, while delivering ultrasound for a therapeutic purpose, ultrasound device 12 may also image tissue of patient 14, e.g., for visualization of a target region, monitoring temperature and/or cavitation to evaluate therapy effectiveness, therapy side effects, and patient safety, or beam aberration correction. Ultrasound device 12 may image during delivery of ultrasound based on reflection of the therapeutic ultrasound by tissue of patient 14, or by interleaving delivery of therapeutic ultrasound with imaging ultrasound.

In some examples, ultrasound device 12 is positioned on an external surface of patient proximate to a particular organ of patient 14, and delivers ultrasound to the organ. The ultrasound delivered by ultrasound device 12 may be configured to modulate nerve tissue at the organ. In the illustrated example, ultrasound device 12 is positioned proximate the spleen 15 of patient 14, and ultrasound device 12 delivers ultrasound to the spleen. The delivered ultrasound may be configured to regulate the autoimmune system of and/or reduce an inflammatory response of patient 14. Modulation of one or more neurons of the sympathetic nervous system at spleen 15, such as the splenic nerve or celiac ganglion, may attenuate an immune response, including an inflammatory immune response. In some example, the delivered ultrasound achieves autoimmune regulation or inflammatory response reduction by modulating at least one of the celiac ganglion (not shown) or the splenic nerve (not shown) of patient 14.

In other examples, ultrasound device 12 may be positioned proximate to other organs of patient 14, and deliver ultrasound configured to modulate nerve tissue at the other organs. Example organs include the kidneys, stomach, and intestines. The location of wearable ultrasound device 12 on patient 14 illustrated in FIG. 1 is merely one example, and wearable ultrasound device 12 may be attached anywhere on patient 14 to facilitate a particular diagnostic or therapeutic function.

Delivery of ultrasound to an organ may result in direct modulation of the activity of nerve tissue innervating the organ and/or indirect modulation of the activity of nerve tissue by modulating tissue that can in turn modulate the nerve tissue. In some examples, one or more of the modulated neurons are efferent neurons. Efferent fibers can be modulated where substantially no afferent fibers are present, for example at the end organ, e.g., spleen, served by the efferent fibers. In the case of the splenic nerve, where approximately 98% of the fibers are efferent, the entire nerve can be modulated without producing an excessive afferent modulation.

In the example of FIG. 1, system 10 also includes sensors 18A and 18B (collectively “sensors 18”), which are implanted in or attached to patient 14. Although two sensors 18 are illustrated in FIG. 1, system 10 may include any number of implanted or wearable sensors 18. Additionally, system 10 includes external sensing devices 19A and 19B (collectively, “external sensing devices 19”) that are not implanted in or attached to patient 14. Although two external sensing devices 19 are illustrated in FIG. 1, system 10 may include any number of external sensing devices 19.

System 10 includes sensors, such as sensors 18 and external sensing devices 19, that are configured to sense one or more physiological parameters of patient. In some examples, the one or more physiological parameters are indicative of at least one of a symptom treatable by the modulation of the nerve tissue of the patient at the organ, or a side effect of the modulation of the nerve tissue of the patient at the organ. In some examples, system 10 modifies the delivery of ultrasound from ultrasound device 12 to the organ, e.g., in a closed loop manner, based on the sensed physiological parameters, e.g., based on changes to the monitored symptoms and/or side effects.

In some examples, the sensors comprise one or more sensors configured to detect a level of one or more substances in a fluid of the patient, such as blood, interstitial fluid, cerebrospinal fluid, intestinal fluid. Examples of substances that may be detected in conjunction with the delivery of ultrasound to the spleen include white blood cells, a comprehensive metabolic panel, a complete blood count, or the levels of one or more cytokines. An inflammatory immune response can be mediated by an inflammatory cytokine cascade and can be alleviated by an anti-inflammatory cytokine cascade.

An increase or decrease in an inflammatory immune response may be detected by measuring an increase or decrease in one or more proinflammatory cytokines. Non-limiting examples of proinflammatory cytokines include tumor necrosis factor (TNF; also known as TNFα or cachectin), interleukin (IL)-1α, IL-1β, IL-2, IL-5, IL-6, IL-8, IL-15, IL-18, interferon γ (IFN-γ); platelet-activating factor (PAF), thromboxane; soluble adhesion molecules; vasoactive neuropeptides; phospholipase A2; plasminogen activator inhibitor (PAI-1); free radical generation; neopterin; CD14; prostacyclin; neutrophil elastase; protein kinase; monocyte chemotactic proteins 1 and 2 (MCP-1, MCP-2); macrophage migration inhibitory factor (MIF), high mobility group box protein 1 (HMGB-1), and other known factors. An increase or decrease of an inflammatory immune response may also be detected by measuring a decrease or increase in one or more anti-inflammatory cytokines. Non-limiting examples of anti-inflammatory cytokines include IL-4, IL-10, IL-17, IL-13, IL-1 alpha, and TNFalpha receptor. It will be recognized that some of proinflammatory cytokines may act as anti-inflammatory cytokines in certain circumstances, and vice-versa. Such cytokines are typically referred to as plieotropic cytokines. An increase or decrease of an inflammatory response may also be detected by measuring changes (baseline versus during therapy delivery, a first point in therapy versus a second point in therapy, etc.) in the presence of other factors involved in an immune response. Non-limiting examples of such other factors include TGF, PDGF, VEGF, EGF, FGF, I-CAM, nitric oxide, and other known factors. In addition, an increase or decrease immune response may be detected by changes in chemokines, such as 6cKine and MIP3beta, and chemokine receptors, including CCR7 receptor. Further, an increase or decrease of an immune response may be measured by changes in immune cell population (upregulated Langerhans cells, dendritic cells, lymphocytes), or immune cell surface co-stimulatory molecules (Major Histocompatibility, CD80, CD86, CD28, CD40). An increase or decrease of an inflammatory response may also be detected by measuring changes in other factors involved in the inflammatory cascade, for example in the signal transduction cascades including factors such as NFκ-B, Egr-1, Smads, toll-like receptors, and MAP kinases. An increase or decrease of an immune response may also be detected by a change in the presence of, or the clearance of, an exogenous antigen believed to have caused an inflammatory response, such as a bacteria, a virus, or a fungus. Further, cell types involved in an immune response, such as Langerhans cells, dendritic cells, T lymphocytes, and B lymphocytes may be detected. In addition, cell surface molecules involved in an immune response, such as major histocompatibility complex (MHC), CD80, CD86, CD28, and CD40 may be detected.

In some examples, sensors 18 may be configured to detect levels of such substances in patient 14, and report them, e.g., to ultrasound device 12, via wired or wireless communication. In other examples, a sample of blood or another patient fluid, e.g., obtained by a clinician, may be analyzed by one of external sensing devices 19. In such examples, the external sensing devices 19 may be configured to detect the level of the substance, e.g., a cytokine assay, and report the level, e.g., to ultrasound device 12, via wired or wireless communication. Examples of wearable sensors 18 that may be configured to detect substance levels in patient fluid include those developed by MC10. Example external sensing devices 19 that may be configured to detect substance levels in patient fluid include a “home lab” test, such as the home lab test described by https://cue.me/#inflammation.

The levels of cytokines and other substances involved in the inflammatory response pathway may be monitored to assess efficacy of, or need for, the ultrasound therapy. White blood cell count may, in addition to indicating efficacy of, or need for the therapy, indicate that an infection has developed as a side effect of the immunosuppressant therapy. Other example physiological parameters of patient 14 that sensors, such as sensors 18 and/or external sensing devices 19, may be configured to detect include blood oxygen level, activity level, heart rate, temperature, respiration rate, or blood pressure. Activity level, heart rate, and/or respiration rate may indicate efficacy of, or need for the therapy by indicating the ability of the patient to engage in physical activity, e.g., as an indirect indicator of joint stiffness, which may be impeded by an inflammatory disorder, such as due to rheumatoid arthritis. Increased temperature, at the location of ultrasound device 12 or another location, may indicate efficacy or need for therapy by indicating inflammation, or may indicate infection as a side effect of the immunosuppressant therapy.

In some examples, ultrasound device 12 includes one or more sensors as described herein. Furthermore, in some examples, ultrasound device 12 uses ultrasound to monitor one or more physiological parameters of patient 14. For example, ultrasound device 12 may obtain an image of spleen 15. Increased size of the spleen may indicate an infection as a side effect of the immunosuppressant therapy. As another example, ultrasound device 12 may monitor temperature of tissue proximate the device, which may indicate inflammation or infection, as described above.

Reflection of delivered ultrasound by a particular tissue varies based on the temperature of the tissue and, consequently, the temperature of tissue can be sensed via ultrasound imaging of the tissue. In some examples, the ultrasound transducers of wearable ultrasound device 12 sense the temperature of the proximate tissue. In some examples, system 10 includes another ultrasound device to sense the temperature of the surrounding tissue based on ultrasound imaging of the proximate tissue. Using ultrasound to sense the temperature of the proximate tissue may facilitate sensing temperature of tissue below the outer surface of patient 14, e.g., a three-dimensional volume of tissue surrounding the target point.

In some examples, wearable ultrasound device 12, or another device of system 10, includes temperatures sensors of any type capable of sensing temperature of tissue. For example, wearable ultrasound device 12 may include one or more temperature sensors, such as thermistors or thermocouples, to sense the temperature of tissue proximate to the target point, e.g., at the skin surface of patient 14. As another example, system 10 may include a temperature sensing device 18 that is separate from wearable ultrasound device 12, and includes one or more temperature sensors configured to sense the temperature of tissue. In some examples, temperature sensing device 18 may include one or more thermal imaging devices, such as infrared cameras or thermometers, to sense the temperature of tissue proximate to the target point.

As illustrated in FIG. 1, system 10 also includes an interface device 16, which may be a computing device having a user interface, e.g., a personal computer, workstation, tablet computing device, or cellular telephone. Interface device 16 is configured to communicate, e.g., via a wired or wireless connection, with wearable ultrasound device 12. Interface device 16 may also be configured to communicate, e.g., via a wired or wireless connection, with implanted or wearable sensors 18 and external sensing devices 19. Interface device 16 may control, e.g., program, wearable ultrasound device 12, sensors 18, and sensing devices 19. Interface device 16 may also receive sensed physiological parameter information from wearable ultrasound device 12, sensors 18, and external sensing devices 19. Although not illustrated in FIG. 1, system 10 may include one or more other remote computing devices connected to interface device 16 via a network, and the one or more remote computing devices may control and/or receive information from wearable ultrasound device 12, sensors 18, and external sensing devices 19 via interface device 16. In some examples, interface device 16 and one or more external sensing devices 19 may be integrated as a single device.

System 10 includes one or more processors or other processing circuitry, e.g., of wearable ultrasound device 12, interface device 16, and/or the one or more remote computing devices, that are configured to control wearable ultrasound device 12, interface device 16, sensors 18, sensing devices 19, or any other ultrasound device, sensor, or any other device described herein to provide the functionality described herein.

For example, one or more processors or other processing circuitry of one or more of these devices may be configured to control ultrasound device 12 to deliver ultrasound during an ambulatory period of patient 14 to modulate the nerve tissue at the organ, e.g., spleen 15, and monitor at least one of a symptom treatable by the neuromodulation or a side effect during the ambulatory period based on one or more physiological parameters sensed by one or more sensors, e.g., sensors 18 and/or sensing devices 19. Delivery of ultrasound during an ambulatory period of patient 14 may entail delivering ultrasound over an extended period of time that is not necessarily confined to a clinic visit or in-clinic procedure, e.g., chronically as opposed to acutely. An ambulatory period of patient 14 may be a number of days, weeks, months, or years. During the ambulatory period, ultrasound device 14 may deliver ultrasound substantially continuously, periodically, or on demand, e.g., initiated or suspended in response to increased or decreased symptoms or side effects. Periodic delivery of ultrasound may be for a number of minutes every one or more hours or days, a number of hours every one or more days.

In some examples, the one or more processors or other processing circuitry are configured to control ultrasound device 12 to modify the ultrasound based on at least one of the symptom or side effect indicated by the sensed physiological parameters. In some examples, interface device 16 includes a user interface configured to present sensed physiological parameters, or other information derived from the physiological parameters that indicates symptoms or side effects, to a user. The user may modify the delivery of ultrasound based on the information.

In some examples, the one or more processors or other processing circuitry are configured to provide closed loop control of the ultrasound based on the at least one of the symptom or side effect. For example, the one or more processors may increase at least one of an intensity, duty cycle, or duration of the ultrasound in response to an increase in the symptom, e.g., inflammation or pro-inflammatory cytokines, or decrease at least one of an intensity, duty cycle, or duration of the ultrasound in response to at least one of a decrease in the symptom or an increase in the side effect. In some examples, the one or more processors are configured to suspend the ultrasound in response to an increase in the side effect, e.g., a physiological parameter indicating infection, such as white blood cell count, spleen size, or temperature.

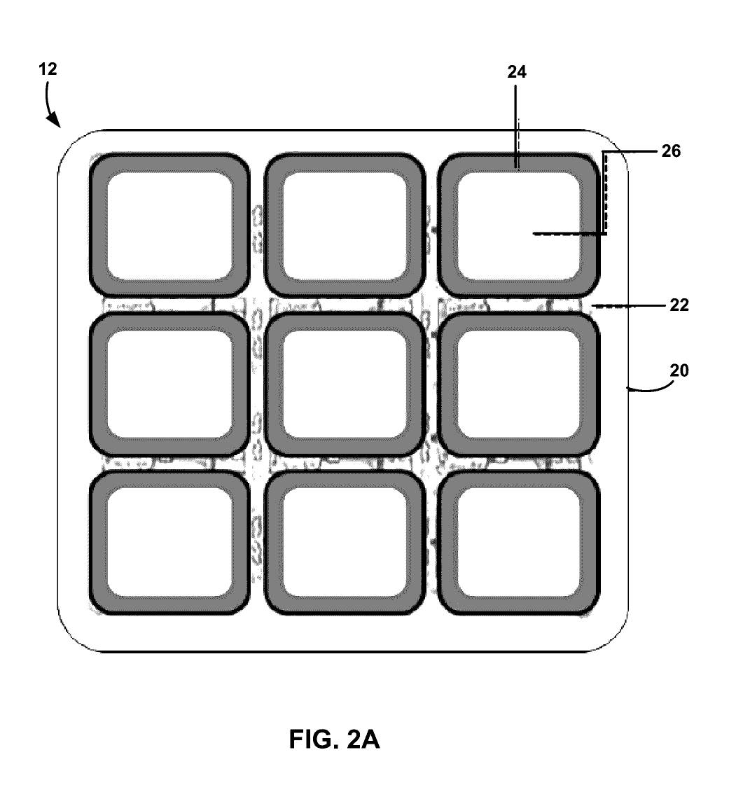

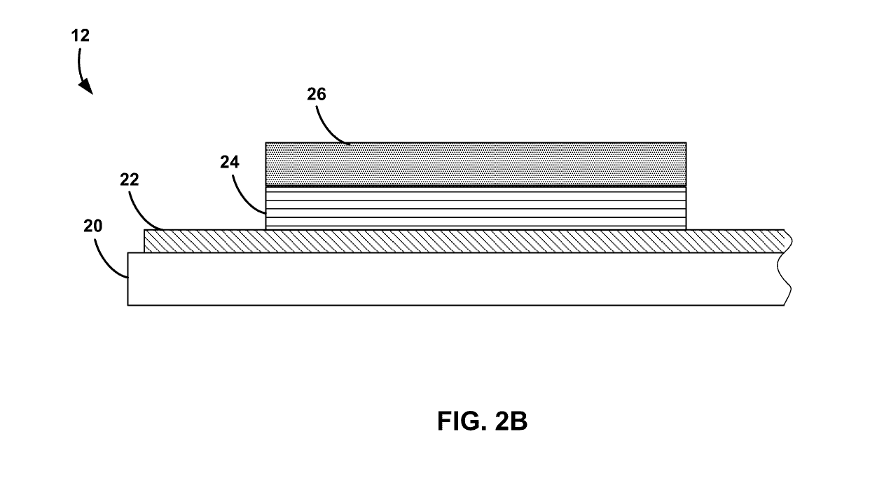

FIGS. 2A and 2B are top-view and side-view diagrams, respectively, illustrating one example configuration of wearable ultrasound device 12. In the example of FIGS. 2A and 2B, wearable ultrasound device 12 includes an adhesive layer 20, a flexible interconnect element 22, a plurality of ultrasound transducers 24 connected to flexible interconnect element 22, and a plurality of power sources 26, e.g., batteries, connected to flexible interconnect element 22. FIG. 2B illustrates one ultrasound transducer 24 and one battery 26. Although not illustrated in FIGS. 2A and 2B, wearable ultrasound device 12 may also include signal generation circuitry, one or more processors or other processing circuitry, sensing circuitry, and communication circuitry, e.g., configured to communicate with interface device 16 (FIG. 1), connected to flexible interconnect element 22.

The components of wearable ultrasound device 12 may be configured, e.g., constructed and arranged, such that wearable ultrasound device 12 is flexible. For example, flexible interconnect element 22 may comprise a flexible circuit, e.g., a flex circuit that electrically connects two or more of the components of wearable ultrasound device 12. Flexible interconnect element 22 and adhesive layer 20 may comprise mechanically compliant materials. Additionally, ultrasound transducers 24 and power sources 26 may be discrete and distributed across wearable ultrasound device 12, e.g., in a two-dimensional array as illustrated in FIG. 2A, which may facilitate flexibility of the wearable ultrasound device. In some examples, signal generation circuitry that drives ultrasound transducers 24 may include flexible driving electronics.

Having a plurality of power sources 26 may also increase the onboard power capacity of wearable ultrasound device 12. In some examples, power sources 26 comprise rechargeable batteries. In such examples, wearable ultrasound device 12 may include a recharge interface, such as a coil for inductive recharging or connector, e.g., universal serial bus (USB), mini-USB, or micro-USB, for wired recharging of power sources 26. In some examples, interface device 16 (FIG. 1) or another device charges power sources 26 of wearable ultrasound device 12,

In some examples, as illustrated by FIG. 2A, each of power sources 26 is associated with a respective one of ultrasound transducers 24. In some examples, each of power sources 26 is attached to the respective ultrasound transducer 24. In such examples, power sources 26 may be configured as a backing material for transducers 24, to tune a frequency of the respective ultrasound transducer. Some ultrasound systems may include a backing material behind the acoustic material to ‘tune’ the frequency. Using power sources 26 as a backing material may reduce or eliminate the need for a dedicated backing material to tune transducers 24, which may in turn reduce the size, e.g., volume, thickness, or weight of the transducers. Various features of power sources 26, such as thickness and mass, may be chosen to tune the ultrasound output parameters, e.g., frequency. In some examples, flexible interconnect element 22 may also be configured as a backing material for ultrasound transducers 24, in addition to, or instead of, power source 26. In some examples, additional electrical components may be affixed, e.g., directly, to the ultrasound material, e.g., during the manufacturing process, and may act as backing material for transducers 24, alone or in combination with other components of device 12.

The relative vertical arrangement of adhesive layer 20, interconnect layer 22, ultrasound transducers 24, and power sources 26 illustrated in FIG. 2B is merely one example. In other examples, interconnect layer 22 may be at least partially between ultrasound transducers 24 and power sources 26, or power sources 26 may be at least partially between interconnect layer 22 and ultrasound transducers 24. In some examples, discrete components, such as ultrasound transducers 24 and power sources 26, may be located at least partially within, e.g., may be at least partially surrounded by, interconnect layer 22 and/or adhesive layer.

Although nine ultrasound transducers 24 and nine power sources 26 are illustrated in FIG. 2A, in other examples, the numbers of transducers and power sources may be different than illustrated, and the number of transducers 24 may be different than the number of power sources. In some examples, there may be at least three, at least nine, at least thirty-two, or at least sixty-four transducers 24 and/or power sources 26. In some examples, power sources 26 may be horizontally adjacent transducers 24. In some examples, one or more power sources 26 may be located anywhere in interconnect element 22 to power transducers 24 (e.g., the signal generation circuitry that drives the transducers) and other components of wearable ultrasound device 12.

Power sources 26 may be connected in series, parallel, or in some series/parallel combination. At least partial series combination may boost voltage of the resulting power source. To improve acoustic coupling and tune the ultrasound, the cavity within the power source case (e.g., a battery case) may substantially free of gas (e.g., free or nearly free), such as by completely filling the space between electrodes with an electrolyte that may be liquid, gel or solid. In some examples, power sources 26 comprise a battery chemistry that does not generate gas during charge/discharge (for example, using a lithium titanate anode) and/or to allow for removal of gas that is usually formed during the initial charge cycle (known in the art as formation) of the cell. The power source encasement may be a metal such as titanium or aluminum or a metal/polymer foil laminate, although other materials can be used in other examples. The performance of power sources 26 as backing material may be configured based on acoustic impedance (density x sound speed), thickness, and attenuation coefficient to reduce reflections.

The piezoelectric material of ultrasound transducers 24 may be, as examples, one or more of lead zirconate titanate (PZT) composite, PZT film, polyvinylidene fluoride (PVDF), which is a plastic with piezoelectric properties, thin-film piezoelectric materials (e.g., either lead-containing or non-lead containing materials), and/or capacitive micromachined ultrasonic transducers (CMUTs). In examples in which power sources 26 and transducers 24 are attached, the ultrasound material may be glued or otherwise bonded to the surface of the power source. In some examples, a metallic housing of a power source 26 may be part of an electrical circuit of wearable ultrasound device 12, e.g., to couple ultrasound material of a transducer 24 to the power source 26, signal generation circuitry for driving the transducer 24, and/or sensing circuitry for processing reflected ultrasound for diagnostic or therapy monitoring purposes.

Adhesive layer 20 attaches wearable ultrasound device 12 to patient 14 (FIG. 1). In some examples, adhesive layer 20 is also configured to provide an acoustic interface between transducers 24 and tissue of patient 14 for ultrasound. In such examples, the adhesive of adhesive layer 20 may be between, e.g., substantially completely fill the space between, each of transducers 24 and a surface of patient 14.

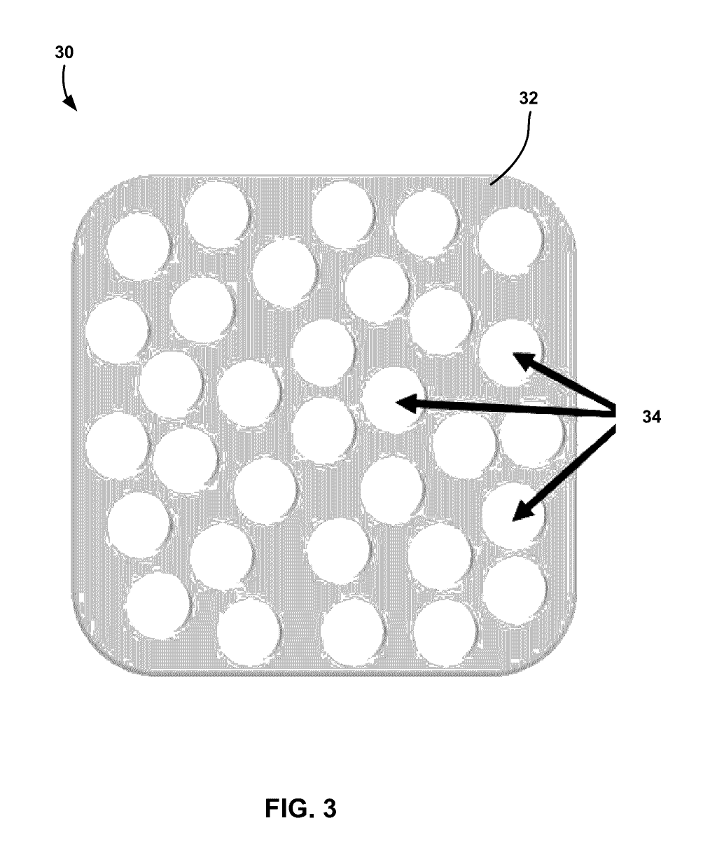

FIG. 3 is a top-view diagram illustrating another example wearable ultrasound device 30. Like wearable ultrasound device 12 of FIGS. 1-2B, wearable ultrasound device 30 includes an interconnect element 32, and a plurality of ultrasound transducers 34 connected to the interconnect element. Although not illustrated in FIG. 3, wearable ultrasound device 30 may also include an adhesive layer, one or more power sources, and other electrical components described above with respect to wearable ultrasound device 12 of FIGS. 1-2B.

Like wearable ultrasound device 12 of FIGS. 1-2B, ultrasound transducers 34 of wearable ultrasound device 30 are connected to and/or distributed on interconnect element 32 in a two-dimensional array. Interconnect element 32 may be completely flexible, include different portions having respectively different flexibilities, or include one or more rigid portions and one or more flexible portions. In other examples, interconnect element 32 may be flexible and connect two or more different regions, each region including one or more ultrasound transducers 24. In this manner, each ultrasound transducer may be flexed with respect to another ultrasound transducer or an array may include rigid and flexible regions such that not all ultrasound transducers may be moved with respect to every other ultrasound transducer. However, unlike ultrasound transducers 24 in FIG. 2A, ultrasound transducers 34 are not necessarily arranged in rows and columns. Ultrasound transducers 34 may be arranged in any suitable way on wearable ultrasound device 30. Ultrasound transducers 34 may be arranged on wearable ultrasound device 30 in a way that, for example, improves ultrasound delivery and/or sensing, e.g., for a particular diagnostic or therapeutic purpose, reduces size and/or increase flexibility of ultrasound device 30, or improves power consumption or heat dissipation by ultrasound device 30.

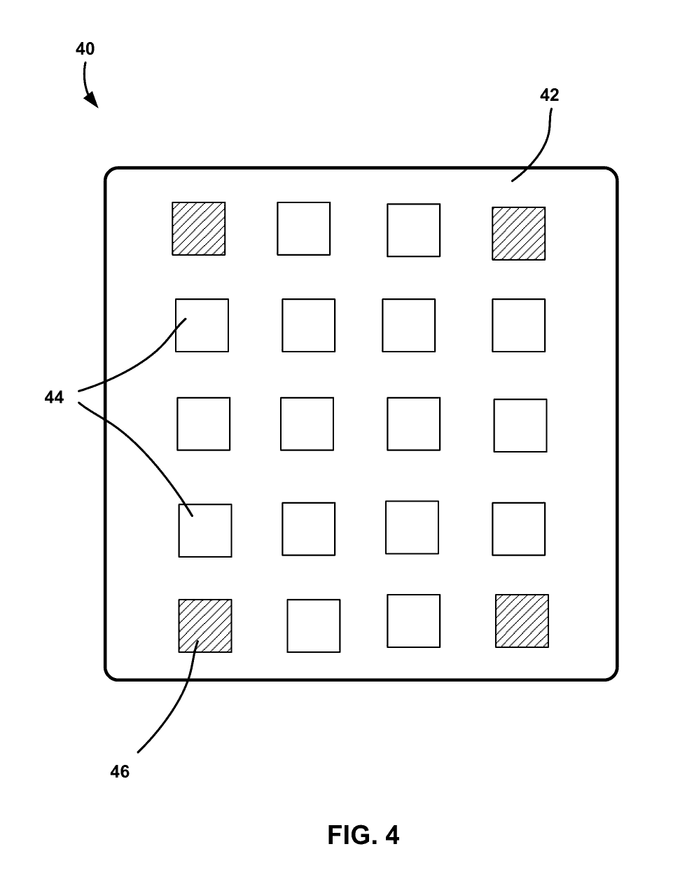

FIG. 4 is a top-view diagram illustrating another example wearable ultrasound device 40. Like wearable ultrasound device 12 of FIGS. 1-2B, wearable ultrasound device 40 includes an interconnect element 42, and a plurality of ultrasound transducers 44 connected to the interconnect element. As illustrated by FIG. 4, wearable ultrasound device 40 additionally includes a plurality of sensors 46 connected to interconnect element 42.

Sensors 46 may be configured to sense or measure any of the physiological parameters described herein, e.g., that indicate a symptom treatable by the delivery of ultrasound to an organ or a side effect of the ultrasound delivery. For example, sensors may be configured to measure heart rate, blood pressure, respiration rate, blood oxygenation, or temperature at the surface of tissue beneath wearable ultrasound device 40.

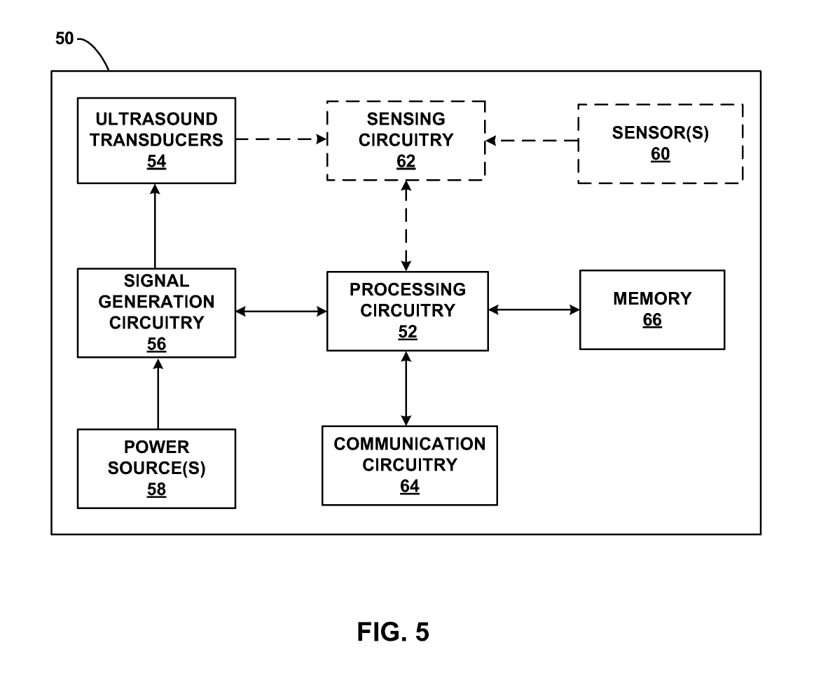

FIG. 5 is a functional block diagram illustrating an example configuration of a wearable ultrasound device 50, which may correspond to any of wearable ultrasound devices 12 (FIGS. 1-2B), 30 (FIG. 3), and 40 (FIG. 4). As illustrated in FIG. 5, ultrasound device 50 includes processing circuitry 52, a plurality of ultrasound transducers 54, signal generation circuitry 56 for driving the ultrasound transducers 54 to deliver ultrasound, and one or more power sources 58 that provide power to the signal generation circuitry 56 for driving transducers 54, as well as providing power to other components ultrasound device 50. Ultrasound transducers 54 and power sources 58 may correspond to any ultrasound transducers, e.g., 24 (FIGS. 2A and 2B), 34 (FIG. 3), or 44 (FIG. 4), and power sources, e.g., power sources 26 (FIGS. 2A and 2B), respectively, described herein.

As illustrated in FIG. 5, ultrasound device 50 may also include communication circuitry 64 and memory 66. Memory 66, as well as other memories described herein, may include any volatile or non-volatile media, such as a random access memory (RAM), read only memory (ROM), non-volatile RAM (NVRAM), electrically erasable programmable ROM (EEPROM), flash memory, and the like. Memory 66 may store computer-readable instructions that, when executed by processing circuitry 52, cause ultrasound device 50 to perform various functions described herein. Processing circuitry 52 may comprise any combination of one or more processors including one or more microprocessors, digital signal processors (DSPs), application specific integrated circuits (ASICs), field programmable gate arrays (FPGAs), or other equivalent integrated or discrete logic circuitry. Accordingly, processing circuitry 52 may include any suitable structure, whether in hardware, software, firmware, or any combination thereof, to perform the functions ascribed herein to processing circuitry 52 and ultrasound device 50.

Processing circuitry 52 is configured to control ultrasound transducers 54 to deliver ultrasound, e.g., for a therapeutic or diagnostic purpose. More particularly, processing circuitry 52 controls signal generation circuitry 56 to generate a signal based on power from power source(s) 58 that drives the ultrasound transducers to deliver ultrasound. Signal generation circuitry 56 may include one or more oscillators configured to generate signals of a desired frequency for the ultrasound, amplification or other circuitry to control the amplitude of the driving signals, as well as switching circuitry to selectively direct the signal to one or more of transducers 54 and/or selectively control the on/off state of individual ones or groups of transducers 54. Some or all of the signal generation circuitry may be respectively associated with certain ones or groups of transducers 54, or shared by all or a subset of transducers 54. Processing circuitry 52 may control ultrasound transducers 54 to deliver ultrasound to a particular depth, region, or point of tissue, with a particular amplitude, by selecting which of transducers 54 is on or driven, and controlling one or more of the amplitude or phase of the driving signal provided to the driven transducers 54 by signal generation circuitry 56. Different active transducers 54 may be driven with different signals, e.g., different amplitudes and/or phases, to target a desired, depth, region, or point of tissue.

In examples in which ultrasound device 50 is configured for diagnostic ultrasound, e.g., to sense spleen size or temperature via diagnostic ultrasound, ultrasound device 50 may include sensing circuitry 62 to selectively, e.g., as controlled by processing circuitry 52, receive and condition electrical signals produced ultrasound transducers 54 as a function of reflected ultrasound, for processing by processing circuitry 52. Sensing circuitry 62 may include one or more switches to control which one or more of transducers 54 are active to sense reflected ultrasound. Ultrasound device 50 may also include one or more physiological parameter sensors 60, which may correspond to any sensors described herein. Sensing circuitry 62 may selectively, e.g., as controlled by processing circuitry 52, receive and condition electrical signals produced sensor(s) 60 as a function of a physiological parameter, for processing by processing circuitry 52. Sensing circuitry 62 may include one or more switches to control which one or more of sensor(s) 60 are active to sense a physiological parameter.

Power source(s) 58 may deliver operating power to various components of ultrasound device 50. Power source(s) 58 may include a small rechargeable or non-rechargeable batteries and a power generation circuit to produce the operating power. Recharging may be accomplished through proximal inductive interaction between a charging device and an inductive charging coil of ultrasound device 50, or a wired connection between the charging device and ultrasound device 50.

Communication circuitry 64 is configured to support wired or wireless communication between ultrasound device 50 and one or more other devices, such as interface device 16, sensors 18, and external sensing devices 19. A user may control the delivery of ultrasound by ultrasound device 50, as well as the collection of diagnostic ultrasound and/or physiological parameter sensing by ultrasound device 50, via communication with processor(s) 52 through communication circuitry 64. In some examples, programs that control the delivery of ultrasound, collection of diagnostic ultrasound, and/or physiological parameter sensing may be stored in memory 66, and executed by processor(s) 52. A user may generate or update such programs, using interface device 16, through communication with ultrasound device 50 via communication circuitry 64. Interface device 16, or another device, may also receive diagnostic ultrasound images or sensed temperatures collected by processing circuitry 52, or any other information generated by processing circuitry 52, via communication circuitry 64. Such information may be stored in memory 66.

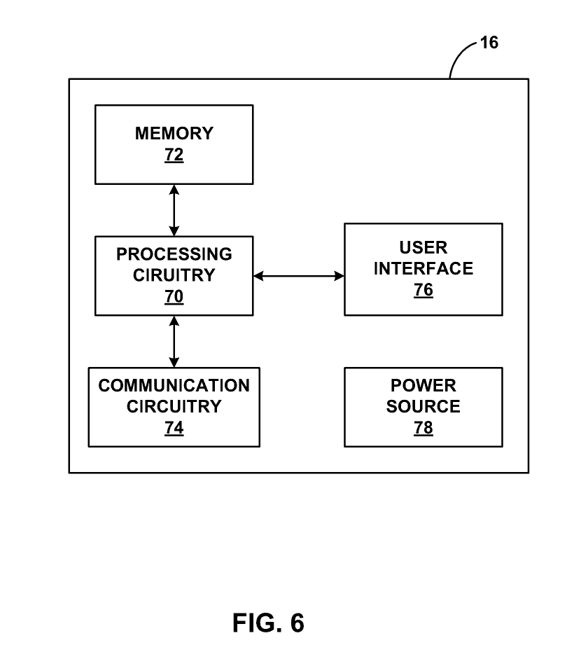

FIG. 6 is a functional block diagram illustrating an example configuration of interface device 16. As illustrated in FIG. 6, interface device 16 includes processing circuitry 70, a memory 72, a communication circuitry 74, a user interface 76, and a power source 78 configured to power the components of interface device 16. Processor 70 controls user interface 76 and communication circuitry 74, and stores and retrieves information and instructions to and from memory 72. Interface device 16 may be referred to as a computing device, as processing circuitry 70 may perform calculations or determinations based on information received from ultrasound device 12 or any other sensor or device described herein. In some examples, interface device 16 may be configured as a mobile computing device comprising one or more software and/or hardware applications configured to perform the functions described herein, such as receiving data from ultrasound device 12 and/or other sensors and/or controlling ultrasound device 12 to obtain ultrasound images and/or deliver ultrasound signals. If interface device 16 is configured to control ultrasound device 12, interface device 16 may be referred to as an external programmer device associated with ultrasound device 12.

Processing circuitry 70 may comprise any combination of one or more processors including one or more microprocessors, DSPs, ASICs, FPGAs, or other equivalent integrated or discrete logic circuitry. Accordingly, processing circuitry 70 may include any suitable structure, whether in hardware, software, firmware, or any combination thereof, to perform the functions ascribed herein to processing circuitry 70 and interface device 16. Memory 72 may include program instructions that, when executed by processor 70, cause processing circuitry 70 and interface device 16 to perform any of the functions ascribed to them herein. Memory 72 may include any volatile or nonvolatile memory, such as RAM, ROM, EEPROM or flash memory.

A user, such as a clinician, other caregiver, or patient 14, may interact with interface device 16 through user interface 76. User interface 76 includes a display, with which processing circuitry 70 may present information, such as information physiological parameters, symptoms treatable by delivery of ultrasound to nerve tissue at on organ, or side effects of such neuromodulation, as described herein, or any other information retrieved from ultrasound device 50. In addition, user interface 76 may include an input mechanism to receive input from the user, though which the user may control or program delivery of ultrasound and or physiological parameter sensing according to any of the techniques described herein.

Communication circuitry 74 is configured for wired or wireless communication with the corresponding communication circuitry 64 of ultrasound device 50, to facilitate user control or programming of the ultrasound device, or retrieval of information from the ultrasound device. Communication circuitry 74 may also be configured for wired or wireless communication with any of sensors 18, external sensing devices 19, or any other device described herein, such as one or more networked computing resources. Interface device 16 may include processing circuitry 70 configured to monitor symptoms and side effects, e.g., based on physiological parameter sensed by sensors 18, sensing devices 19, sensors 46, and sensors 60. Processing circuitry 70 may be configured to control an ultrasound device to modify the delivery of ultrasound based on changes, or lack thereof, in the one or more symptoms or side effects over time.

In some examples, user interface 76 may include an input mechanism for a user, e.g., patient 14 or a caregiver, to input information regarding the patient's perception of symptoms and side effects. Processing circuitry described herein, e.g., processing circuitry 70, may consider such user input information alone, or in conjunction with physiological parameters sensed by sensors, and control an ultrasound device to modify the delivery of ultrasound based on the analysis.

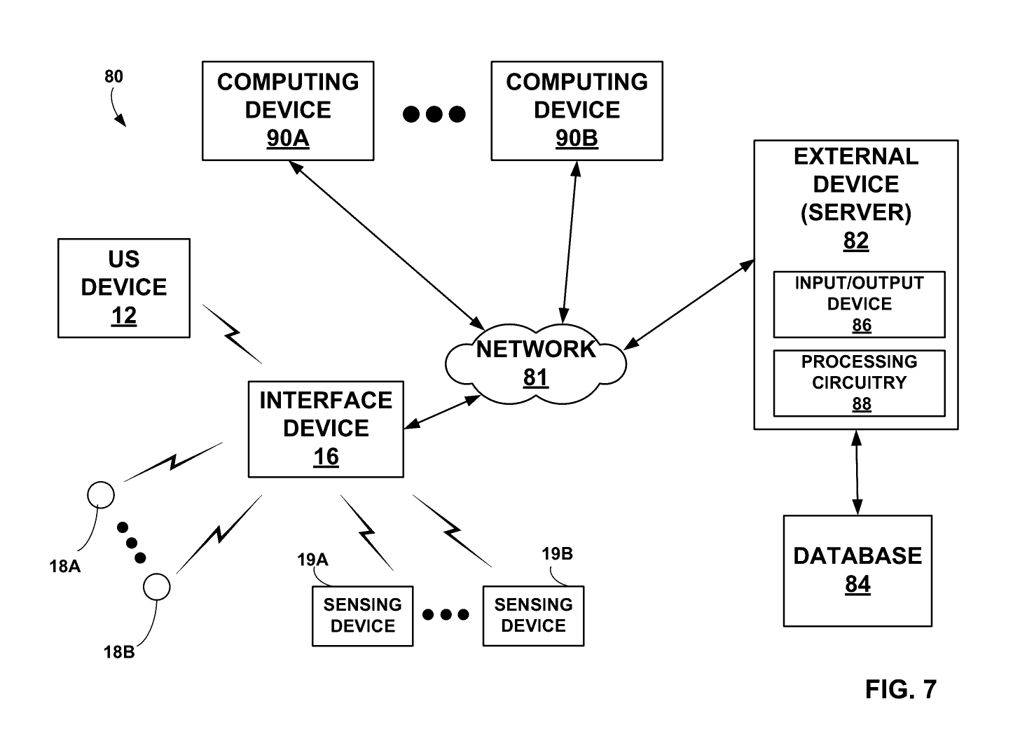

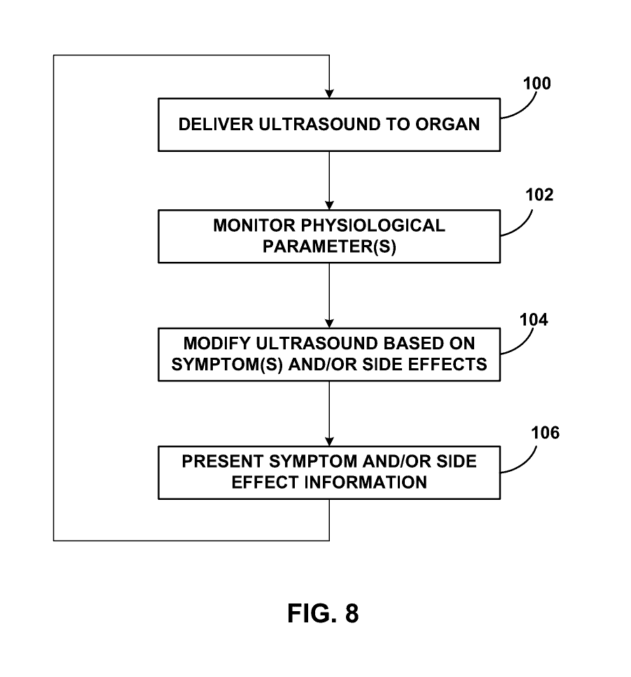

FIG. 7 is a block diagram illustrating another example system 80 for delivering ultrasound to a patient and monitoring the patient. Like system 10 of FIG. 1, system 80 includes flexible, wearable ultrasound (US) device 12, interface device 16, sensors 18, and external sensing devices 19. Example system 80 of FIG. 8 includes a number of additional computing resources accessible via network 81, such as a remote or external serve 82, a database 84, and computing devices 90A and 90B (collectively “computing devices 90”). Although two computing devices 90 are illustrated in FIG. 7, system 80 may include any number of computing devices 90. External server 82 includes an input/output device 86, e.g., user interface, as well as processing circuitry 88. Although not illustrated in FIG. 7, computing devices 90 may similarly include user interfaces and processing circuitry.

Server 82 is configured to receive information regarding physiological parameters, symptoms, side effects, the delivery of ultrasound therapy, or any other information regarding patient 14 or the devices of system 80, via network 81. Server 82 may be configured to store such information, collected over time, in database 84. The information stored in database 84 may form a longitudinal record of the symptoms and side effects experienced by patient 14, and the course of therapy delivered to the patient. Although the example of FIG. 7 illustrates interface device 16 acting as a portal to network 81 for ultrasound device 12, sensors 18, and external sensing device 19, any one or more of these may be configured to communicate directly with network 81.

Computing devices 90 may be associated with a variety of users, such as physicians, clinicians, caregivers, or patients. Computing devices 90 may communicate with server 82 via network 81 to allow users to retrieve and view the data stored in database 84, or to communicate with interface device 16, ultrasound device 12, sensors 18, or external sensing devices 19. In some examples, users may user computing devices 90 to remotely program, modify, or control the delivery of ultrasound by ultrasound device 12, e.g., by interacting with the ultrasound device or interface device 16 via server 82 and/or network 80. In some examples, a user, e.g., patient 14, may provide information regarding the patient's perception of symptoms and side effects via a computing device 90, which may be stored in database 84, and/or used by one or more processors of system 80 to control delivery of ultrasound therapy. Any of server 82 and computing devices 90 may include one or more processors configured to provide any of the functionality described herein, such as monitoring symptoms and side effects, and controlling, e.g., providing closed loop control, of ultrasound therapy.

FIG. 8 is a flow diagram illustrating an example method for delivering ultrasound to an organ and monitoring a patient during the delivery of the ultrasound. The method of FIG. 8 will be described as being performed by the components of ultrasound device 50, such as processing circuitry 52. However, the method of FIG. 8 may be performed by any ultrasound devices described herein (e.g., ultrasound devices 12, 30, 40, or 50), interface device 16, computing device 90A, networked server 82, or any combination thereof, e.g., any combination of the processing circuitry of such devices. Combinations of these devices may be referred to as a distributed system where certain functions are distributed between two or more devices of the system.

According to the example method of FIG. 8, an ultrasound device, e.g., ultrasound device 12, 30, 40, or 50 described herein, delivers ultrasound to an organ, e.g., the spleen, to modulate nerve tissue at the organ during an ambulatory period of the patient (100). The ultrasound device may operate according to a set of therapy parameters that define the ultrasound energy (e.g., frequency, amplitude, pulse width, etc.) that is selected to target one or more nerves at the organ. This ambulatory period of the patient may be over the period of several hours, days, weeks, months, or even years. Processing circuitry 52 monitors one or more physiological parameters of the patient that are indicative of at least one symptom treatable by, or side effect of, the neuromodulation delivered during the ambulatory period based on information received from one or more sensors (102). For example, processing circuitry 52 may be configured to periodically measure values of one or more physiological parameters and compare the values to one or more thresholds that may be indicative of a symptom or side effect of the neuromodulation. Processing circuitry 52 modifies the ultrasound, e.g., in a closed loop, based on the monitored symptoms and/or side effects (104). In one example, processing circuitry 52 may change one or more parameters defining the ultrasound in response to detecting that a physiological parameter exceeds a respective threshold. Processing circuitry 52 also provides current and/or longitudinal information (e.g., information indicative of past detected physiological parameters) regarding the symptom and/or side effect to a user, e.g., via interface device 16 or computing device 90 (106). For example, interface device 16 may present the current and/or longitudinal information as one or more of numerical data, graphical data, trend information, or any other data forms. In some examples, interface device 16 may deliver one or more alerts in response to detecting that a physiological parameter exceeds a threshold and/or the ultrasound therapy is modified or will be modified.

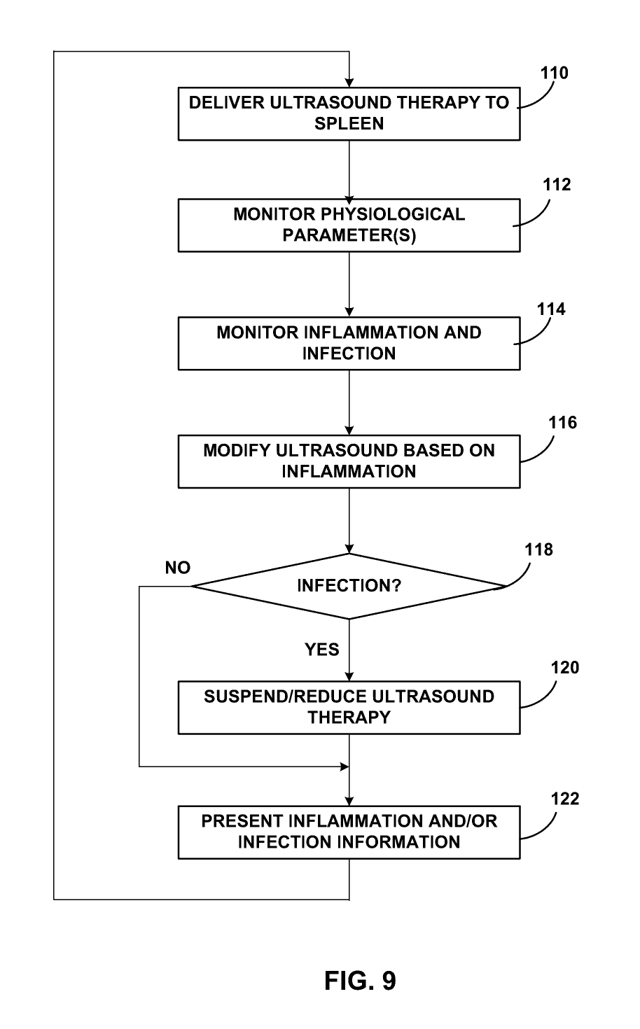

FIG. 9 is a flow diagram illustrating an example method for delivering ultrasound to the spleen and monitoring a patient during the delivery of the ultrasound. The method of FIG. 9 will be described as being performed by the components of ultrasound device 50, such as processing circuitry 52. However, the method of FIG. 9 may be performed by any ultrasound devices described herein (e.g., ultrasound devices 12, 30, 40, or 50), interface device 16, computing device 90A, networked server 82, or any combination thereof, e.g., any combination of the processing circuitry of such devices. Combinations of these devices may be referred to as a distributed system where certain functions are distributed between two or more devices of the system.

According to the example method of FIG. 9, an ultrasound device, e.g., ultrasound device 12, 30, 40, or 50, positioned on a surface of the patient proximate to the spleen delivers ultrasound to the spleen to modulate nerve tissue at the spleen during an ambulatory period of the patient (110). The ultrasound is configured to at least one of regulate the autoimmune system of the patient, or reduce an inflammation response of the patient, and may be configured to modulate at least one of the Celiac ganglion or the Splenic nerve of the patient.

Processing circuitry 5 monitors one or more physiological parameters via one or more sensors (112), and monitors for inflammation or infection of the patient during the ambulatory period based on one or more physiological parameters sensed by one or more sensors as described herein (114). For example, inflammation may be indicated by a level of one or more pro-inflammatory (or anti-inflammatory) cytokines, or other substances involved in mediating the autoimmune or inflammatory response, in blood or other fluid of the patient, as described herein. As other examples, inflammation may be indicated by temperature or swelling evidence in ultrasound images of tissues collected by the ultrasound device, or reduced patient activity. Infection may be indicated by increased temperature, white blood cell count, or spleen size as determined based on ultrasound images collected by the ultrasound device.

Processing circuitry 52 modifies the ultrasound, e.g., in a closed loop, based on the inflammation (116). For example, processing circuitry 52 may be configured to increase at least one of an intensity, duty cycle, or duration of the ultrasound in response to increased inflammation or increased inflammatory response, e.g., as indicated by increased levels or pro-inflammatory cytokines or decreased levels of anti-inflammatory cytokines, or decreased activity level or increased swelling and temperature. Processing circuitry 52 may be configured to decrease at least one of an intensity, duty cycle, or duration of the ultrasound in response to at least one of decreased inflammation or decreased inflammatory response.

Processing circuitry 52 is also configured to determine whether immunosuppression resulting from the delivery of the ultrasound therapy to the spleen may have resulted in an infection (118). If so, processing circuitry 52 may control the ultrasound device to suspend or reduce the delivery of ultrasound (120). The suspension may be for a fixed period of time, until reactivated by a clinician, or until the monitored physiological parameters indicate absence or improvement of the infection. Physiological parameters indicative of infection may include white blood cell counts, spleen size, and temperature, as described herein. Processing circuitry 52 also presents current and/or longitudinal information inflammation and/or infection to a user, e.g., via interface device 16 or computing device 90 (122).

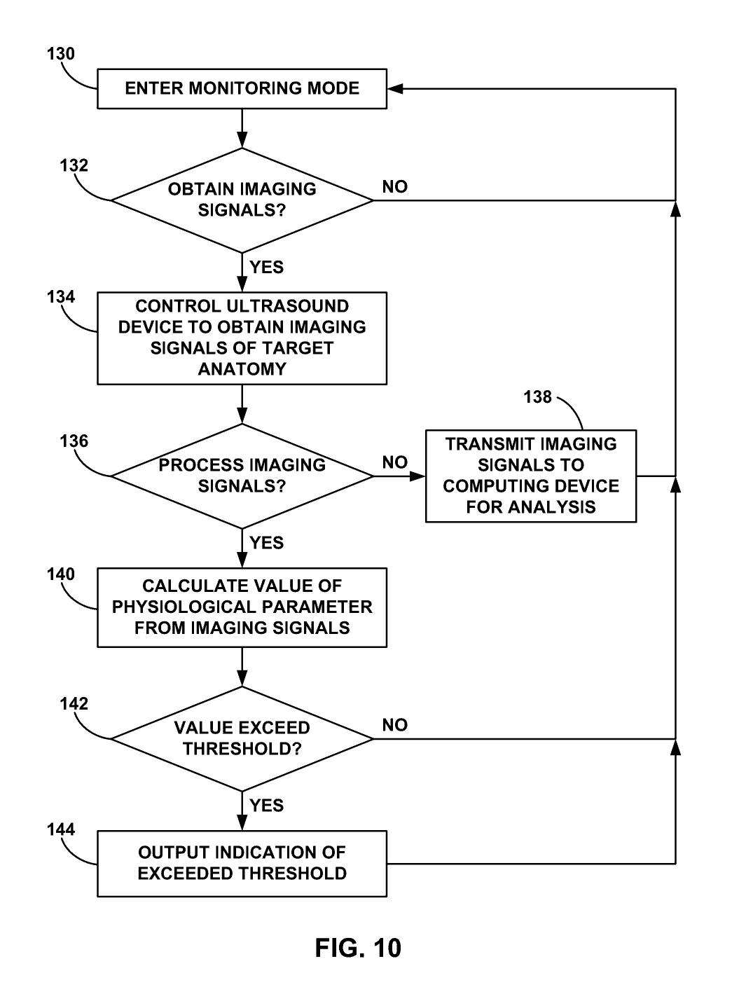

FIG. 10 is a flow diagram illustrating an example process for determining changes to a physiological parameter based on ultrasound imaging signals. The process of FIG. 10 will be described as being performed by the components of ultrasound device 50, such as processing circuitry 52. However, the process of FIG. 10 may be performed by any ultrasound devices described herein (e.g., ultrasound devices 12, 30, 40, or 50), interface device 16, computing device 90A, networked server 82, or any combination thereof. Combinations of these devices may be referred to as a distributed system where certain functions are distributed between two or more devices of the system.

As shown in FIG. 10, processing circuitry 52 enters a monitoring mode (130). Processing circuitry 52 may enter monitoring mode 130 as a default during therapy delivery or in response to receiving a request from a user (e.g., the patient or a clinician). In other examples, processing circuitry 52 may enter the monitoring mode in response to detecting a change in a physiological parameter of the patient that was detected as part of another function executing by processing circuitry 52 or detected by another device or sensor in communication with ultrasound device 50. If processing circuitry 52 determines that no ultrasound imaging should be obtained (“NO” branch of block 132), processing circuitry 52 continues to operate in the monitoring mode (130).

If processing circuitry 52 is scheduled to obtain imaging signals using one or more ultrasound transducers 54 (“YES” branch of block 132), processing circuitry 52 then controls signal generator 56 and sensing circuitry 62 to obtain one or more imaging signals of target anatomy of the patient (134). For example, processing circuitry 52 may control signal generator 56 to send one or more drive signals to a subset or all of ultrasound transducers 54 configured to target a particular portion of patient anatomy (e.g., an organ such as the spleen, heart, or intestines) with ultrasound signals and control sensing circuitry 62 to receive the reflected signals via one or more of ultrasound transducers 54. Sensing circuitry 62 may transmit the received signals to processing circuitry 52 for processing and analysis. The imaging signals may be representative of one or more physiological parameters of the patient, and a value of the respective physiological parameters (e.g., a size of an organ or structure, tissue density, tissue elasticity or stiffness, blood flow rate) may be calculated from the imaging signals by processing circuitry 52, for example.

Wearable ultrasound device 50 may or may not be configured to process or analyze the imaging signals from sensing circuitry 62. If processing circuitry 52 is not instructed to process the imaging signals (“NO” branch of block 136), processing circuitry 52 may control communication circuitry 64 to send some or all of the imaging signals to another computing device (e.g., interface device 16 or networked server 82) for processing. In this manner, the other computing device may be configured to perform at least one of the remaining steps 140, 142, and 144 described further below.

If processing circuitry 52 is instructed to process the imaging signals according to the operating instructions stored in memory 66 (“YES” branch of block 136), processing circuitry 52 proceeds to calculate the value of a physiological parameter from the imaging signals (140). Processing circuitry 52 also compares the calculated value to a respective threshold to determine if the value exceeds the threshold (142). If processing circuitry 52 determines that the value does not exceed the threshold (“NO” branch of block 142), processing circuitry 52 stores the value in memory 66 and continue to monitor physiological parameters of the patient. If processing circuitry 52 determines that the value does exceed the threshold (“YES” branch of block 142), processing circuitry 52 outputs the indication of the exceeded threshold (144). Processing circuitry 52 may output the indication to an external computing device (e.g., interface device 16) for presentation to a user, to memory 66, and/or for use in modulating ultrasound therapy delivered by ultrasound device 50.

In this manner, the process of FIG. 10 may include generating, by sensing circuitry 62 of wearable ultrasound device 50, ultrasound imaging signals indicative of one or more physiological parameters over a period of time. The period of time may be relatively short when used for relatively immediate (e.g., on the order of seconds, minutes, or hours) information during an emergency event or when used as feedback to modulate ultrasound therapy delivery. The period of time may also be on the order of days, weeks, months, or even years. These longer monitoring periods may be referred to as longitudinal monitoring made possible by a wearable ultrasound device such as any of ultrasound devices 12, 30, 40, or 50. Ultrasound device 50, or any other device described herein, may determine, based on the ultrasound imaging signals generated by sensing circuitry 62, for example, that a value of the physiological parameter has exceeded a respective threshold and output the indication of the determination.

In some examples, the threshold may be a predetermined value stored in a memory of a device (e.g., ultrasound device 50 or interface device 16) and indicative of an abnormality in anatomy represented by the ultrasound imaging signals. For example, the threshold may be a certain size (e.g., distance, cross-sectional area, or volume) of an organ or other structure. The threshold may alternatively be a blood flow rate value or any other value. In other examples, the threshold may be one of a plurality of thresholds indicating respective levels of the value. In some examples, the system may calculate the threshold based on a plurality of previously determined values of the physiological parameter (e.g., a median, mean, rolling mean, or weighted mean) that is used to identify changes to or variations of the physiological parameter over time.

In some examples, the outputted indication may include an alert. A user interface (e.g., user interface 76 of interface device 16 or user interface of wearable ultrasound device 50) associated with the wearable ultrasound device may display the alert to a user. The alert may include a request for a user to schedule a clinic visit and/or instructions for the patient wearing the ultrasound device to at least one of change a behavior or take medication.

As described herein, processing circuitry 52, for example, may control the signal generator 62 based on the indication that the value exceeded the threshold. In one example, processing circuitry 52 may control signal generation circuit 62 to generate the drive signal to the one or more ultrasound transducers 54 to modulate nerve tissue that at least partially controls the physiological parameter. Alternatively, processing circuitry 52 may control signal generation circuit 62 to suspend generation of the drive signal until a subsequent value of the physiological parameter no longer exceeds the threshold.

The physiological parameter may include an organ size, an organ location, tissue structure size or location, a tissue density, tissue elasticity or stiffness, a blood flow rate, a temperature. The organ may be a spleen, the physiological parameter may be a size of the spleen, and the value may be representative of a magnitude of the size of the spleen. In some cases, an increase in the size of the spleen is indicative of an acute infection. Depending on the ultrasound energy being delivered, processing circuitry 52 may modulate ultrasound delivery based whether an infection has been identified. For example, processing circuitry 52 may control signal generation circuitry 56 to generate drive signals that cause the one or more ultrasound transducers 54 to deliver a reduced inhibition of immune response or suspend generation of drive signals in response to identifying the increased size of the spleen. This action may allow the spleen to fight a potential infection through normal physiological pathways. Alternatively, processing circuitry 52 may control signal generation circuitry 56 to generate drive signals that cause the one or more ultrasound transducers 54 to deliver an increased promotion of immune response or initiate generation of drive signal in response to identifying the increased size of the spleen. This action may help the spleen to respond to the potential infection. These inhibition or promotion of immune response may depend on whether the ultrasound energy is delivered to a nerve that promotes or inhibits immune response from the spleen.

In other examples, the change in the size of the spleen may be used as a biomarker indicative of ultrasound modulation of spleen function by ultrasound therapy delivered by the wearable ultrasound device. In other words, since ultrasound therapy delivered to the spleen may modulate spleen function that changes the size of the spleen, processing circuitry 52 may use this change, or new spleen size during ultrasound therapy, as a baseline when attempting to identify further changes in spleen size that may be due to infection. In other words, processing circuitry 52 or another device may be configured to determine an active-therapy baseline size of the spleen based on the change in the size of the spleen during ultrasound therapy delivery from the wearable ultrasound device, wherein the active-therapy baseline is different than a non-therapy baseline size of the spleen during an absence of ultrasound therapy delivery from the wearable ultrasound device.

The techniques described in this disclosure, may be implemented, at least in part, in hardware, software, firmware or any combination thereof. For example, various aspects of the techniques may be implemented within one or more processors, including processing circuitry such as one or more microprocessors, DSPs, ASICs, FPGAs, or any other equivalent integrated or discrete logic circuitry, as well as any combinations of such components, embodied in programmers, such as clinician or patient programmers, medical devices, or other devices.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processors, such as one or more DSPs, ASICs, FPGAs, general-purpose microprocessors, or other equivalent integrated or discrete logic circuitry. Accordingly, the term “processor,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

### Example 1

A system comprises a flexible ultrasound device configured to be attached to an external surface of a patient proximate to an organ of the patient, wherein the flexible ultrasound device comprises: a flexible interconnect element; a plurality of ultrasound transducers connected to the flexible interconnect element; one or more power sources connected to the flexible interconnect element; and signal generation circuitry powered by the one or more power sources and connected to the flexible interconnect element, wherein the signal generation circuitry is configured to generate a signal that drives one or more of the ultrasound transducers to deliver an ultrasound signal to the organ, the ultrasound signal configured to modulate nerve tissue of the patient at the organ. The system further comprises: one or more sensors configured to sense one or more physiological parameters of the patient, the one or more physiological parameters indicative of at least one of a symptom treatable by the modulation of the nerve tissue of the patient at the organ, or a side effect of the modulation of the nerve tissue of the patient at the organ; and processing circuitry. The processing circuitry is configured to: control the signal generation circuitry to generate the signal and drive the one or more ultrasound transducers during an ambulatory period of the patient to modulate the nerve tissue at the organ; and monitor the least one of the symptom or the side effect during the ambulatory period based on the one or more physiological parameters.

### Example 2

The system of example 1, wherein the processing circuitry is configured to control the signal generation circuitry to modify the ultrasound based on the at least one of the symptom or side effect.

### Example 3

The system of example 2, wherein the processing circuitry is configured to provide closed loop control of the ultrasound based on the at least one of the symptom or side effect.

### Example 4

The system of example 2 or 3, wherein the processing circuitry is configured to increase at least one of an intensity, duty cycle, or duration of the ultrasound in response to an increase in the symptom.

### Example 5

The system of any of examples 2 to 4, wherein the processing circuitry is configured to decrease at least one of an intensity, duty cycle, or duration of the ultrasound in response to at least one of a decrease in the symptom or an increase in the side effect.

### Example 6

The system of any of examples 2 to 5, wherein the processing circuitry is configured to suspend the ultrasound in response to an increase in the side effect.

### Example 7

The system of any of examples 1 to 6, wherein the organ comprises the spleen of the patient, and the ultrasound is configured to at least one of regulate the autoimmune system of the patient, or reduce an inflammation response of the patient.

### Example 8

The system of example 7, wherein the one or more ultrasound transducers are configured to deliver the ultrasound to modulate at least one of the Celiac ganglion or the Splenic nerve of the patient.

### Example 9

The system of example 7 or 8, wherein the sensors comprise one or more sensors configured to detect one or more substances in blood of the patient, wherein the one or more physiological parameters comprise a level of one or more substances in the blood.

### Example 10

The system of example 9, wherein one or more substances in the blood comprise one or more of: white blood cells; a comprehensive metabolic panel; a complete blood count; oxygen; or one or more cytokines.

### Example 11

The system of example 10, wherein the one or more cytokines comprise one or more pro-inflammatory cytokines.

### Example 12

The system of example 11, wherein a level of the one or more pro-inflammatory cytokines above a threshold is a symptom treatable by the modulation of the nerve tissue of the patient at the organ, and a level of white blood cells below a threshold is a side effect of the modulation of the nerve tissue of the patient at the organ.

### Example 13

The system of any of examples 7 to 12, wherein the one or more physiological parameters comprise one or more of: activity level; heart rate; temperature; respiration rate; or blood pressure.

### Example 14

The system of example 13, wherein activity level below a threshold is a symptom treatable by the modulation of the nerve tissue of the patient at the organ, and temperature above a threshold is a side effect of the modulation of the nerve tissue of the patient at the organ.

### Example 15

The system of any of claims 7 to 14, wherein the one or more sensors comprises one or more of the ultrasound transducers configured to obtain an image of the patient, wherein the one or more physiological parameters comprise one or more of inflammation or spleen size determined based on the image, wherein increased inflammation is a symptom treatable by the modulation of the nerve tissue of the patient at the organ, and increase spleen size is a side effect of the modulation of the nerve tissue of the patient at the organ.

### Example 16

The system of any of examples 1 to 15, wherein the one or more sensors comprise one or more sensors connected to the flexible interconnect element of the flexible device.

### Example 17

The system of any of examples 1 to 16, wherein the one or more sensors comprise one or more sensors implanted in or attached to the patient.

### Example 18

The system of any of examples 1 to 17, further comprising an external medical device comprising one or more of the sensors.

### Example 19

The system of any of examples 1 to 18, wherein the processing circuitry comprises processing circuitry of the flexible ultrasound device coupled to the flexible interconnect element.

### Example 20

The system of example 19, wherein the flexible device comprises communication circuitry coupled to the flexible interconnect element, the communication circuitry configured for wireless communication with at least one of the one or more sensors.

### Example 21

The system of any of examples 1 to 20, further comprising at least one of an external interface device or a remote server, wherein the processing circuitry comprises processing circuitry of the at least one of the interface device or the remote server, wherein the flexible device comprises communication circuitry coupled to the flexible interconnect element, the communication circuitry configured for wireless communication with the at least one of the external interface device or the remote server.

### Example 22

The system of example 21, wherein the at least one of the external interface device or a remote server comprises a user interface configured to at least one of: receive user input indicating a perception of the patient of the symptom or the side effect, wherein the at least one processor of the external interface device is configured to control the delivery of ultrasound by the flexible device based on the user input; or present values of the one or more physiological parameters over time to a user.

### Example 23

A method of delivering ultrasound with a flexible ultrasound device configured to be attached to an external surface of a patient proximate to an organ of the patient, the method comprising: delivering ultrasound from the flexible ultrasound device to the organ during an ambulatory period of the patient, the ultrasound configured to modulate nerve tissue of the patient at the organ; sensing, via one or more sensors, one or more physiological parameters of the patient during the ambulatory period, the one or more physiological parameters indicative of at least one of a symptom treatable by the modulation of the nerve tissue of the patient at the organ, or a side effect of the modulation of the nerve tissue of the patient at the organ; and monitoring, via processing circuitry, the least one of the symptom or the side effect during the ambulatory period based on the one or more physiological parameters.

### Example 24

The method of example 23, further comprising modifying, by the processing circuitry, the ultrasound based on the at least one of the symptom or side effect.

### Example 25

The method of example 24, wherein modifying the ultrasound based on the at least one of the symptom or side effect comprising providing closed loop control of the ultrasound based on the at least one of the symptom or side effect.

### Example 26

The method of example 24 or 25, wherein modifying the ultrasound comprises increasing at least one of an intensity, duty cycle, or duration of the ultrasound in response to an increase in the symptom.

### Example 27

The method of any of examples 24 to 26, wherein modifying the ultrasound comprises decreasing at least one of an intensity, duty cycle, or duration of the ultrasound in response to at least one of a decrease in the symptom or an increase in the side effect.

### Example 28

The method of any of examples 24 to 27, wherein modifying the ultrasound comprises suspending the ultrasound in response to an increase in the side effect.

### Example 29

The method of any of examples 23 to 28, wherein delivering ultrasound from the flexible device to the organ comprises delivering ultrasound from the flexible device to the spleen of the patient, wherein the ultrasound is configured to at least one of regulate the autoimmune system of the patient, or reduce an inflammation response of the patient.

### Example 30

The method of example 29, wherein delivering ultrasound from the flexible device to the spleen comprises modulating at least one of the Celiac ganglion or the Splenic nerve of the patient.

### Example 31

The method of example 29 or 30, wherein sensing one or more physiological parameters of the patient comprises detecting a level of one or more substances in blood of the patient.

### Example 32

The method of example 31, wherein one or more substances in the blood comprise one or more of: white blood cells; a comprehensive metabolic panel; a complete blood count; oxygen; or one or more cytokines.

### Example 33

The method of example 32, wherein the one or more cytokines comprise one or more pro-inflammatory cytokines.

### Example 34

The method of example 33, wherein a level of the one or more pro-inflammatory cytokines above a threshold is a symptom treatable by the modulation of the nerve tissue of the patient at the organ, and a level of white blood cells below a threshold is a side effect of the modulation of the nerve tissue of the patient at the organ.

### Example 35

The method of any of examples 29 to 34, wherein sensing one or more physiological parameters of the patient comprises sensing one or more of: activity level; heart rate; temperature; respiration rate; or blood pressure.

### Example 36

The method of example 35, wherein activity level below a threshold is a symptom treatable by the modulation of the nerve tissue of the patient at the organ, and temperature above a threshold is a side effect of the modulation of the nerve tissue of the patient at the organ.

### Example 37

The method of any of examples 29 to 36, wherein the one or more sensors comprises one or more of ultrasound transducers of the flexible device configured to obtain an image of the patient, wherein sensing the one or more physiological parameters comprise sensing one or more of inflammation or spleen size via the image, wherein increased inflammation is a symptom treatable by the modulation of the nerve tissue of the patient at the organ, and increase spleen size is a side effect of the modulation of the nerve tissue of the patient at the organ.

### Example 38

A system comprising means to perform any of the methods of example 23 to 37.

### Example 39

A computer-readable storage medium having instructions stored thereon that, when executed by one or more programmable processors, cause the processors to perform any of the methods of examples 23 to 37.

### Example 40

A system for delivering ultrasound with a flexible ultrasound device configured to be attached to an external surface of a patient proximate to an organ of the patient, the system comprising: means for delivering ultrasound from the flexible ultrasound device to the organ during an ambulatory period of the patient, the ultrasound configured to modulate nerve tissue of the patient at the organ; means for sensing one or more physiological parameters of the patient during the ambulatory period, the one or more physiological parameters indicative of at least one of a symptom treatable by the modulation of the nerve tissue of the patient at the organ, or a side effect of the modulation of the nerve tissue of the patient at the organ; and means for monitoring the least one of the symptom or the side effect during the ambulatory period based on the one or more physiological parameters.

### Example 41

A computer-readable storage medium comprising program instructions that, when executed by processing circuitry, cause the processing circuitry to: control a flexible ultrasound device to deliver ultrasound to an organ of a patient during an ambulatory period of the patient, the ultrasound configured to modulate nerve tissue of the patient at the organ, and the flexible ultrasound device configured to be attached to an external surface of the patient proximate to the organ of the patient; control one or more sensors to sensing one or more physiological parameters of the patient during the ambulatory period, the one or more physiological parameters indicative of at least one of a symptom treatable by the modulation of the nerve tissue of the patient at the organ, or a side effect of the modulation of the nerve tissue of the patient at the organ; and monitor the least one of the symptom or the side effect during the ambulatory period based on the one or more physiological parameters.

### Example 42

A method comprising: generating, by sensing circuitry of a wearable ultrasound device, ultrasound imaging signals indicative of a physiological parameter of a patient over a period of time. The wearable ultrasound device comprises: a flexible interconnect element; a plurality of ultrasound transducers connected to the flexible interconnect element; one or more power sources connected to the flexible interconnect element; signal generation circuitry powered by the one or more power sources and connected to the flexible interconnect element, wherein the signal generation circuitry is configured to generate a drive signal that drives one or more ultrasound transducers of the ultrasound transducers to deliver an ultrasound signal to target anatomy; and the sensing circuitry, wherein the sensing circuitry is connected to one or more of the plurality of ultrasound transducers and the flexible interconnect element, and wherein, for at least one ultrasound transducer of the plurality of ultrasound transducers, the sensing circuitry is configured to generate the imaging signals as a function of reflected ultrasound sensed by the at least one ultrasound transducer. The method further comprises: determining, based on the ultrasound imaging signals, that a value of the physiological parameter has exceeded a threshold during the period of time; and outputting an indication of the determination.

### Example 43

The method of example 42, wherein determining the value of the physiological parameter comprises determining, by processing circuitry of the wearable ultrasound device, the value of the physiological parameter.

### Example 44

The method of any of examples 42 and 43, further comprising transmitting, by communication circuitry of the wearable ultrasound device, physiological data representative of the generated imaging signals to a computing device distinct from the wearable ultrasound device, wherein: determining the value of the physiological parameter comprises determining, by the computing device, the value of the physiological parameter; and outputting the indication of the determination comprises outputting, by the computing device, the indication of the determination.

### Example 45

The method of any of examples 42 to 44, wherein determining that the value of the physiological parameter has exceeded the threshold comprises: processing at least some of the ultrasound imaging signals to calculate respective values of the physiological parameter; comparing the respective values of the physiological parameter to the threshold; and determining, based on the comparison, that at least one of the respective values of the physiological parameter exceeds the threshold.

### Example 46

The method of any of examples 42 to 45, wherein the threshold comprises a predetermined value stored in a memory and indicative of an abnormality in anatomy represented by the ultrasound imaging signals.

### Example 47

The method of any of examples 42 to 46, wherein the value of the physiological parameter is a current value, and wherein the method further comprises calculating the threshold based on a plurality of previously determined values of the physiological parameter, the previously determined values being determined prior to the current value.

### Example 48

The method of any of examples 42 to 47, wherein the indication comprises an alert.

### Example 49

The method of example 48, wherein outputting the alert comprises outputting, by a user interface associated with the wearable ultrasound device and for display to a user, the alert.

### Example 50

The method of example 49, wherein at least one of a mobile computing device or an external programmer for the wearable ultrasound device comprises the user interface.

### Example 51

The method of any of examples 48 to 50, wherein the alert comprises a request for a user to schedule a clinic visit.

### Example 52

The method of any of examples 48 to 51, wherein the alert comprises instructions for a patient wearing the ultrasound device to at least one of change a behavior or take medication.

### Example 53

The method of any of examples 42 to 52, further comprising controlling, by processing circuitry of the wearable ultrasound device and based on the indication that the value exceeded the threshold, the signal generation circuit to generate the drive signal to the one or more ultrasound transducers to modulate nerve tissue that at least partially controls the physiological parameter.

### Example 54

The method of any of examples 42 to 53, further comprising controlling, by processing circuitry of the wearable ultrasound device and based on the indication that the value exceeded the threshold, the signal generation circuit to suspend generation of the drive signal until a subsequent value of the physiological parameter no longer exceeds the threshold.

### Example 55

The method of any of examples 42 to 54, wherein the physiological parameter comprises an organ size, an organ location, a tissue density, a tissue elasticity, a blood flow rate, a temperature.

### Example 56

The method of example 55, wherein the organ comprises a spleen.

### Example 57

The method of example 56, wherein the physiological parameter is a size of the spleen and the value is representative of a magnitude of the size of the spleen.

### Example 58

The method of example 57, wherein an increase in the size of the spleen is indicative of an acute infection.

### Example 59

The method of example 58, further comprising: controlling the signal generation circuitry of the wearable ultrasound device to generate drive signals that cause the one or more ultrasound transducers to deliver ultrasound signals configured to inhibit immune response; and responsive to determining that the size of the spleen is increased, controlling the signal generation circuitry to one of generate drive signals that cause the one or more ultrasound transducers to one of deliver a reduced inhibition of immune response or suspend generation of drive signals.

### Example 60

The method of claim 56, further comprising: controlling the signal generation circuitry of the wearable ultrasound device to generate drive signals that cause the one or more ultrasound transducers to deliver ultrasound signals configured to promote immune response; and responsive to determining that the size of the spleen is increased, controlling the signal generation circuitry to one of generate drive signals that cause the one or more ultrasound transducers to deliver one of an increased promotion of immune response or initiate generation of drive signals.

### Example 61

The method of any of examples 57 to 60, wherein a change in the size of the spleen is indicative of ultrasound modulation of spleen function by ultrasound therapy delivered by the wearable ultrasound device.

### Example 62

The method of example 61, further comprising determining an active-therapy baseline size of the spleen based on the change in the size of the spleen during ultrasound therapy delivery from the wearable ultrasound device, wherein the active-therapy baseline is different than a non-therapy baseline size of the spleen during an absence of ultrasound therapy delivery from the wearable ultrasound device.

### Example 63

The method of any of examples 42 to 62, wherein the indication that the value of the physiological parameter has exceeded the threshold represents an infection in a patient wearing the wearable ultrasound device.

### Example 64

A system configured to perform any of the methods of claims 42 to 63, wherein the system comprises the wearable ultrasound device.

### Example 65

The system of example 64, further comprising a computing device configured to at least one of control the wearable ultrasound device or receive data from the wearable ultrasound device.

### Example 66

A computer-readable storage medium having instructions stored thereon that, when executed by processing circuitry, cause the processing circuitry to perform any of the methods of claims 42 to 63.

### Example 67

A wearable ultrasound device comprising: a flexible interconnect element; a plurality of ultrasound transducers connected to the flexible interconnect element; one or more power sources connected to the flexible interconnect element; signal generation circuitry powered by the one or more power sources and connected to the flexible interconnect element, wherein the signal generation circuitry is configured to generate a drive signal that drives one or more ultrasound transducers of the ultrasound transducers to deliver an ultrasound signal to target anatomy; sensing circuitry connected to one or more of the plurality of ultrasound transducers and the flexible interconnect element and configured to generate ultrasound imaging signals indicative of a physiological parameter of a patient over a period of time; and processing circuitry configured to: control the signal generation circuitry and the sensing circuitry; determine, based on the ultrasound imaging signals, that a value of the physiological parameter has exceeded a threshold; and output an indication of the determination.

### Example 68

The wearable ultrasound device of example 67, further comprising communication circuitry configured to transmit the indication of the determination that the value to a computing device distinct from the wearable ultrasound device.

### Example 69

The wearable ultrasound device of example 68, wherein the computing device comprises a user interface configured to deliver the indication to a user.

### Example 70

The wearable ultrasound device of any of examples 67 to 69, wherein the processing circuitry is configured to determine that the value of the physiological parameter has exceeded the threshold by: processing at least some of the ultrasound imaging signals to calculate respective values of the physiological parameter; comparing the respective values of the physiological parameter to the threshold; and determining, based on the comparison, that at least one of the respective values of the physiological parameter exceeds the threshold.

### Example 71

The wearable ultrasound device of any of examples 67 to 70, further comprising a memory, wherein the threshold comprises a predetermined value stored in the memory and indicative of an abnormality in anatomy represented by the ultrasound imaging signals.

### Example 72

The wearable ultrasound device of any of examples 67 to 71, wherein the value of the physiological parameter is a current value, and wherein the processing circuitry is further configured to calculate the threshold based on a plurality of previously determined values of the physiological parameter, the previously determined values being determined prior to the current value.

### Example 73

The wearable ultrasound device of any of examples 67 to 72, wherein the indication comprises an alert for display to user via a user interface associated with the wearable ultrasound device.

### Example 74

The wearable ultrasound device of any of examples 67 to 73, wherein the processing circuitry is configured to control, based on the indication that the value exceeded the threshold, the signal generation circuit to generate the drive signal to the one or more ultrasound transducers to modulate nerve tissue that at least partially controls the physiological parameter.

### Example 75

The wearable ultrasound device of any of examples 67 to 74, wherein the processing circuitry is configured to control, based on the indication that the value exceeded the threshold, the signal generation circuit to suspend generation of the drive signal until a subsequent value of the physiological parameter no longer exceeds the threshold.

### Example 76

The wearable ultrasound device of any of examples 67 to 75, wherein the physiological parameter comprises an organ size, an organ location, a tissue density, a tissue elasticity, a blood flow rate, a temperature.

### Example 77

The wearable ultrasound device of example 76, wherein the organ comprises a spleen.

### Example 78

The wearable ultrasound device of example 77, wherein the physiological parameter is a size of the spleen and the value is representative of a magnitude of the size of the spleen.

### Example 79

The wearable ultrasound device of example 78, wherein an increase in the size of the spleen is indicative of an acute infection.

### Example 80

The wearable ultrasound device of example 79, wherein the processing circuitry is configured to: control the signal generation circuitry to generate drive signals that cause the one or more ultrasound transducers to deliver ultrasound signals configured to inhibit immune response; and responsive to determining that the size of the spleen is increased, control the signal generation circuitry to one of generate drive signals that cause the one or more ultrasound transducers to deliver a reduced inhibition of immune response or suspend generation of drive signals.

### Example 81

The wearable ultrasound device of example 79, wherein the processing circuitry is configured to: control the signal generation circuitry to generate drive signals that cause the one or more ultrasound transducers to deliver ultrasound signals configured to promote immune response; and responsive to determining that the size of the spleen is increased, control the signal generation circuitry to one of generate drive signals that cause the one or more ultrasound transducers to deliver an increased promotion of immune response or initiate generation of drive signals.

### Example 82

The wearable ultrasound device of any of examples 78 to 79, wherein a change in the size of the spleen is indicative of ultrasound modulation of spleen function by ultrasound therapy delivered by the wearable ultrasound device.

### Example 83

The wearable ultrasound device of example 82, wherein the processing circuitry is configured to determine an active-therapy baseline size of the spleen based on the change in the size of the spleen during ultrasound therapy delivery from the wearable ultrasound device, wherein the active-therapy baseline is different than a non-therapy baseline size of the spleen during an absence of ultrasound therapy delivery from the wearable ultrasound device.

### Example 84

The wearable ultrasound device of any of examples 67 to 83, wherein the indication that the value of the physiological parameter has exceeded the threshold represents an infection in a patient wearing the wearable ultrasound device.

Various examples have been described. These and other examples may be within the scope of the following claims.

What is claimed is:

1. A system comprising:
a flexible ultrasound device configured to be attached to an external surface of a patient proximate to an organ of the patient, wherein the flexible ultrasound device comprises:
a flexible interconnect element;
a plurality of ultrasound transducers connected to the flexible interconnect element;
one or more power sources connected to the flexible interconnect element; and
signal generation circuitry powered by the one or more power sources and connected to the flexible interconnect element, wherein the signal generation circuitry is configured to generate a signal that drives one or more of the ultrasound transducers to deliver an ultrasound signal to the organ, the ultrasound signal configured to modulate nerve tissue of the patient at the organ;
one or more sensors configured to sense one or more physiological parameters of the patient, the one or more physiological parameters indicative of at least one of a symptom treatable by the modulation of the nerve tissue of the patient at the organ, or a side effect of the modulation of the nerve tissue of the patient at the organ;
processing circuitry configured to:
control the signal generation circuitry to generate the signal and drive the one or more ultrasound transducers during an ambulatory period of the patient to modulate the nerve tissue at the organ; and
monitor the least one of the symptom or the side effect during the ambulatory period based on the one or more physiological parameters.

2. The system of claim 1, wherein the processing circuitry is configured to control the signal generation circuitry to modify the ultrasound based on the at least one of the symptom or side effect.

3. The system of claim 2, wherein the processing circuitry is configured to provide closed loop control of the ultrasound based on the at least one of the symptom or side effect.

4. The system of claim 2, wherein the processing circuitry is configured to at least one of:
increase at least one of an intensity, duty cycle, or duration of the ultrasound in response to an increase in the symptom;
decrease at least one of an intensity, duty cycle, or duration of the ultrasound in response to at least one of a decrease in the symptom or an increase in the side effect; or
suspend the ultrasound in response to an increase in the side effect.

5. The system of claim 1, wherein the organ comprises the spleen of the patient, and the ultrasound is configured to at least one of regulate the autoimmune system of the patient, or reduce an inflammation response of the patient.

6. The system of claim 5, wherein the one or more ultrasound transducers are configured to deliver the ultrasound to modulate at least one of the Celiac ganglion or the Splenic nerve of the patient.

7. The system of claim 5, wherein the sensors comprise one or more sensors configured to detect one or more substances in blood of the patient, wherein the one or more physiological parameters comprise a level of one or more substances in the blood.

8. The system of claim 7, wherein one or more substances in the blood comprise one or more of:
white blood cells;
a comprehensive metabolic panel;
a complete blood count;
oxygen; or
one or more cytokines.

9. The system of claim 8, wherein the one or more cytokines comprise one or more pro-inflammatory cytokines, and wherein a level of the one or more pro-inflammatory cytokines above a threshold is a symptom treatable by the modulation of the nerve tissue of the patient at the organ, and a level of white blood cells below a threshold is a side effect of the modulation of the nerve tissue of the patient at the organ.

10. The system of claim 5, wherein the one or more physiological parameters comprise one or more of:
activity level;
heart rate;
temperature;
respiration rate; or
blood pressure.

11. The system of claim 10, wherein activity level below a threshold is a symptom treatable by the modulation of the nerve tissue of the patient at the organ, and temperature above a threshold is a side effect of the modulation of the nerve tissue of the patient at the organ.

12. The system of claim 5, wherein the one or more sensors comprises one or more of the ultrasound transducers configured to obtain an image of the patient, wherein the one or more physiological parameters comprise one or more of inflammation or spleen size determined based on the image, wherein increased inflammation is a symptom treatable by the modulation of the nerve tissue of the patient at the organ, and increase spleen size is a side effect of the modulation of the nerve tissue of the patient at the organ.

13. The system of claim 1, wherein the one or more sensors comprise one or more sensors connected to the flexible interconnect element of the flexible device.

14. The system of claim 1, wherein the one or more sensors comprise one or more sensors implanted in or attached to the patient.

15. The system of claim 1, further comprising an external medical device comprising one or more of the sensors.

16. The system of claim 1, wherein the processing circuitry comprises processing circuitry of the flexible ultrasound device coupled to the flexible interconnect element.

17. The system of claim 16, wherein the flexible device comprises communication circuitry coupled to the flexible interconnect element, the communication circuitry configured for wireless communication with at least one of the one or more sensors.

18. The system of claim 1, further comprising at least one of an external interface device or a remote server, wherein the processing circuitry comprises processing circuitry of the at least one of the interface device or the remote server, wherein the flexible device comprises communication circuitry coupled to the flexible interconnect element, the communication circuitry configured for wireless communication with the at least one of the external interface device or the remote server.

19. The system of claim 18, wherein the at least one of the external interface device or a remote server comprises a user interface configured to at least one of:
receive user input indicating a perception of the patient of the symptom or the side effect, wherein the at least one processor of the external interface device is configured to control the delivery of ultrasound by the flexible device based on the user input; or
present values of the one or more physiological parameters over time to a user.

20. A method of delivering ultrasound with a flexible ultrasound device configured to be attached to an external surface of a patient proximate to an organ of the patient, the method comprising:
delivering ultrasound from the flexible ultrasound device to the organ during an ambulatory period of the patient, the ultrasound configured to modulate nerve tissue of the patient at the organ;
sensing, via one or more sensors, one or more physiological parameters of the patient during the ambulatory period, the one or more physiological parameters indicative of at least one of a symptom treatable by the modulation of the nerve tissue of the patient at the organ, or a side effect of the modulation of the nerve tissue of the patient at the organ; and
monitoring, via processing circuitry, the least one of the symptom or the side effect during the ambulatory period based on the one or more physiological parameters.

21. The method of claim 20, further comprising modifying, by the processing circuitry, the ultrasound based on the at least one of the symptom or side effect.

22. The method of claim 21, wherein modifying the ultrasound based on the at least one of the symptom or side effect comprising providing closed loop control of the ultrasound based on the at least one of the symptom or side effect.

23. The method of claim 21, wherein modifying the ultrasound comprises at least one of:
increasing at least one of an intensity, duty cycle, or duration of the ultrasound in response to an increase in the symptom;
decreasing at least one of an intensity, duty cycle, or duration of the ultrasound in response to at least one of a decrease in the symptom or an increase in the side effect; or
suspending the ultrasound in response to an increase in the side effect.

24. The method of claim 20, wherein delivering ultrasound from the flexible device to the organ comprises delivering ultrasound from the flexible device to the spleen of the patient, wherein the ultrasound is configured to at least one of regulate the autoimmune system of the patient, or reduce an inflammation response of the patient.

25. The method of claim 24, wherein delivering ultrasound from the flexible device to the spleen comprises modulating at least one of the Celiac ganglion or the Splenic nerve of the patient.

26. The method of claim 24, wherein sensing one or more physiological parameters of the patient comprises detecting a level of one or more substances in blood of the patient.

27. The method of claim 26, wherein one or more substances in the blood comprise one or more of:
white blood cells;
a comprehensive metabolic panel;
a complete blood count;
oxygen; or
one or more cytokines.

28. The method of claim 27, wherein the one or more cytokines comprise one or more pro-inflammatory cytokines, and wherein a level of the one or more pro-inflammatory cytokines above a threshold is a symptom treatable by the modulation of the nerve tissue of the patient at the organ, and a level of white blood cells below a threshold is a side effect of the modulation of the nerve tissue of the patient at the organ.

29. The method of claim 24, wherein sensing one or more physiological parameters of the patient comprises sensing one or more of:
activity level;
heart rate;
temperature;
respiration rate; or
blood pressure.

30. The method of claim 29, wherein activity level below a threshold is a symptom treatable by the modulation of the nerve tissue of the patient at the organ, and temperature above a threshold is a side effect of the modulation of the nerve tissue of the patient at the organ.

31. The method of claim 24, wherein the one or more sensors comprises one or more of ultrasound transducers of the flexible device configured to obtain an image of the patient, wherein sensing the one or more physiological parameters comprise sensing one or more of inflammation or spleen size via the image, wherein increased inflammation is a symptom treatable by the modulation of the nerve tissue of the patient at the organ, and increase spleen size is a side effect of the modulation of the nerve tissue of the patient at the organ.

32. A system for delivering ultrasound with a flexible ultrasound device configured to be attached to an external surface of a patient proximate to an organ of the patient, the system comprising:
means for delivering ultrasound from the flexible ultrasound device to the organ during an ambulatory period of the patient, the ultrasound configured to modulate nerve tissue of the patient at the organ;
means for sensing one or more physiological parameters of the patient during the ambulatory period, the one or more physiological parameters indicative of at least one of a symptom treatable by the modulation of the nerve tissue of the patient at the organ, or a side effect of the modulation of the nerve tissue of the patient at the organ; and
means for monitoring the least one of the symptom or the side effect during the ambulatory period based on the one or more physiological parameters.

33. A computer-readable storage medium comprising program instructions that, when executed by processing circuitry, cause the processing circuitry to:
control a flexible ultrasound device to deliver ultrasound to an organ of a patient during an ambulatory period of the patient, the ultrasound configured to modulate nerve tissue of the patient at the organ, and the flexible ultrasound device configured to be attached to an external surface of the patient proximate to the organ of the patient;
control one or more sensors to sensing one or more physiological parameters of the patient during the ambulatory period, the one or more physiological parameters indicative of at least one of a symptom treatable by the modulation of the nerve tissue of the patient at the organ, or a side effect of the modulation of the nerve tissue of the patient at the organ; and
monitor the least one of the symptom or the side effect during the ambulatory period based on the one or more physiological parameters.
